SEQUENCE LISTING (32 sequences)

SEQ ID NO: 1 - protein, 642 aa, Artificial sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQPLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 2 - protein, 622 aa, Artificial sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDVVKQPLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 3 - protein, 626 aa, Artificial sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDGGSGGSGGSLHLMYVAAAAFVLLFFAGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 4 - protein, 508 aa, Artificial sequence
  Synthetic construct
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDVVKQPLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 5 - protein, 624 aa, Artificial sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDEPPPPAQLHFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 6 - protein, 1244 aa, Artificial sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDVVKQPLHFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGSMALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDVVKQPLHFMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 7 - protein, 273 aa, Artificial sequence
  Synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 8 - protein, 159 aa, Artificial sequence
  Synthetic construct
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVS

SEQ ID NO: 9 - protein, 129 aa, Artificial sequence
  Synthetic construct
KTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQP

SEQ ID NO: 10 - protein, 113 aa, Artificial sequence
  Synthetic construct
KTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDGGSGGSGGS

SEQ ID NO: 11 - protein, 113 aa, Artificial sequence
  Synthetic construct
KTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDEPPPPAQLH

SEQ ID NO: 12 - protein, 24 aa, Artificial sequence
  Synthetic construct
LHLMYVAAAAFVLLFFVGCGVLLS

SEQ ID NO: 13 - protein, 22 aa, Artificial sequence
  Synthetic construct
FMYVAAAAFVLLFFVGCGVLLS

SEQ ID NO: 14 - protein, 5 aa, Artificial sequence
  Synthetic construct
RKRRR

SEQ ID NO: 15 - protein, 211 aa, Artificial sequence
  Synthetic construct
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 16 - protein, 10 aa, Artificial sequence
  Synthetic construct
EQKLISEEDL

SEQ ID NO: 17 - protein, 21 aa, Artificial sequence
  Synthetic construct
MALPVTALLLPLALLLHAARP

SEQ ID NO: 18 - DNA, 20 bp, Artificial sequence
  Synthetic construct
ggagcactgtcctccgaacg

SEQ ID NO: 19 - protein, 109 aa, Artificial sequence
  Synthetic construct
KTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDVVKQP

SEQ ID NO: 20 - protein, 111 aa, Artificial sequence
  Synthetic construct
KTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDVVKQPLH

SEQ ID NO: 21 - protein, 24 aa, Artificial sequence
  Synthetic construct
LHLMYVAAAAFVLLFFAGCGVLLS

SEQ ID NO: 22 - protein, 4 aa, Artificial sequence
  Synthetic construct
  MISC_FEATURE (2)..(3): X can be any amino acid residue
  MISC_FEATURE (4)..(4): X represents a hydrophobic residue such as Leu, Ile, Val, Phe, Trp, Tyr, Val, Met, and Pro
PXXX

SEQ ID NO: 23 - protein, 5 aa, Artificial sequence
  Synthetic construct
  MISC_FEATURE (2)..(3): X can be any amino acid residue
  MISC_FEATURE (4)..(4): X represents a hydrophobic residue such as Leu, Ile, Val, Phe, Trp, Tyr, Val, Met, and Pro
  MISC_FEATURE (5)..(5): X is Ser or Thr
PXXXX

SEQ ID NO: 24 - protein, 6 aa, Artificial sequence
  Synthetic construct
  MISC_FEATURE (2)..(2): X is either Leu or Gln
PXGMTS

SEQ ID NO: 25 - protein, 5 aa, Artificial sequence
  Synthetic construct
  MISC_FEATURE (2)..(2): X is either Leu or Gln
PXGMT

SEQ ID NO: 26 - protein, 3 aa, Artificial sequence
  Synthetic construct
VGR

SEQ ID NO: 27 - protein, 7 aa, Artificial sequence
  Synthetic construct
ENLYTQS

SEQ ID NO: 28 - protein, 5 aa, Artificial sequence
  Synthetic construct
DDDDK

SEQ ID NO: 29 - protein, 4 aa, Artificial sequence
  Synthetic construct
LVPR

SEQ ID NO: 30 - protein, 250 aa, Artificial sequence
  Synthetic construct
  MISC_FEATURE (1)..(250): Bases 1 to 250 may be present or absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 31 - protein, 250 aa, Artificial sequence
  Synthetic construct
  MISC_FEATURE (1)..(250): Bases 1-250 may be present or absent
SGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGG

SEQ ID NO: 32 - protein, 200 aa, Artificial sequence
  Synthetic construct
  MISC_FEATURE (1)..(200): X is any amino acid
  MISC_FEATURE (1)..(200): Bases 1-200 may be present or absent
GGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXSGGXS

(19) United States
(12) Patent Application Publication
(10) Pub. No.: US 2022/0348628 A1
(43) Pub. Date: Nov. 3, 2022

(54) NOVEL RECEPTORS HAVING A FIBRONECTIN REPEAT FOR LIGAND-DEPENDENT TRANSCRIPTIONAL REGULATION

(71) Applicant: The Regents of the University of California, Oakland, CA (US)

(72) Inventors: Kole T. ROYBAL, San Francisco, CA (US); Raymond LIU, San Francisco, CA (US); Iowis ZHU, San Francisco, CA (US)

(21) Appl. No.: 17/762,687

(22) PCT Filed: Sep. 23, 2020
(86) PCT No.: PCT/US2020/052267

§ 371 Date: Mar. 22, 2022

Related U.S. Application Data
(60) Provisional application No. 62/905,266, filed on Sep. 24, 2019.

(51) Int. Cl. C07K 14/705; C07K 16/30; C07K 16/28; C07K 16/22; A61K 35/17; A61K 45/06

(52) U.S. Cl. CPC C07K 14/705; C07K 16/3069; C07K 16/3092; C07K 16/3084; C07K 16/2878; C07K 16/2866; C07K 16/2803; C07K 16/30; C07K 16/22; C07K 16/28; C07K 16/2851; A61K 35/17; A61K 45/06; C07K 2317/622; C07K 2319/00; A61K 38/00

(57) ABSTRACT

The present disclosure generally relates to, among other things, a new class of receptors engineered to modulate transcriptional regulation in a ligand-dependent manner Particularly, the new receptors, even though derived from Notch and Robo, do not require the Notch or Robo regulatory regions previously believed to be necessary for the functioning of the receptors. The disclosure also provides compositions and methods useful for producing such receptors, nucleic acids encoding same, host cells genetically modified with the nucleic acids, as well as methods for modulating an activity of a cell and/or for the treatment of various health conditions such as diseases (e.g., cancers).

### CROSS-REFERENCE TO RELATED APPLICATIONS

This application claims priority to U.S. Provisional Patent Application No. 62/905,266, filed Sep. 24, 2019, the disclosure of which is incorporated by reference herein in its entirety, including any drawings.

### STATEMENT REGARDING FEDERALLY SPONSORED R&D

This invention was made with government support under grant no. OD025751 awarded by The National Institutes of Health. The government has certain rights in the invention.

### INCORPORATION OF THE SEQUENCE LISTING

This application contains a Sequence Listing, which is hereby incorporated by reference in its entirety. The accompanying Sequence Listing text file, named “048536_656001WO_Sequence_Listing_ST25.txt,” was created on Sep. 23, 2020 and is 60 KB.

### FIELD

The present disclosure relates generally to new synthetic cellular receptors that bind cell-surface ligands and have selectable specificities and activities. The disclosure also provides compositions and methods useful for producing such receptors, nucleic acids encoding same, host cells genetically modified with the nucleic acids, as well as methods for modulating an activity of a cell and/or for the treatment of various health conditions such as diseases (e.g., cancers).

### BACKGROUND

An important problem limiting the development of engineered cell therapies in humans is the regulation of therapeutic gene expression to reduce or eliminate interactions causing significant side effects on administration of chimeric antigen receptor T cells (CAR-T) such as, for example, off target activity, on target-off tumor activity (i.e., wherein the CAR-T target is also found on normal cells outside the tumor), and inability to modulate or turn off CAR-T activity when needed. A possible solution to these problems is to use a synthetic receptor that is capable of modifying gene expression and/or cellular behavior.

Notch receptors are transmembrane proteins that mediate cell-cell contact signaling and play a central role in development and other aspects of cell-to-cell communication, e.g. communication between two contacting cells, in which one contacting cell is a “receiver” cell and the other contacting cell is a “sender” cell. Notch receptors expressed in a receiver cell recognize their ligands (e.g., the delta/serrate/lag, or “DSL” family of proteins), expressed on a sending cell. The engagement of notch and delta on these contacting cells leads to two-step proteolysis of the notch receptor that ultimately causes the release of the intracellular portion of the receptor from the membrane into the cytoplasm. Notch has a metalloprotease cleavage site (denoted “S2”), which is normally protected from cleavage by the Notch negative regulatory region (NRR), which consists of three LIN-12-Notch repeat (LNR) modules and a heterodimerization domain (HD). It is believed that this proteolysis is regulated by the force exerted by the sending cell: the DSL ligand pulls on the Notch receptor, and changes the conformation of the NRR, exposing the metalloprotease site. The newly-exposed cut site is cleaved by a constitutively active protease, releasing the extracellular binding portion and negative regulatory region of the receptor. Release of the ligand binding portion of the receptor in turn exposes another cleavage site (denoted “S3”), which is cleaved by gamma secretase within the cell membrane and releases the nuclear homing intracellular domain. W. R. Gordon et al., Dev Cell (2015) 33:729-36. This released domain alters receiver cell behavior by functioning as a transcriptional regulator. Notch receptors are involved in and are required for a variety of cellular functions during development and are important for the function of a vast number of cell-types across species.

Examples of existing first-generation synthetic derivatives of Notch receptors, which are often referred to as “SynNotch receptors”, employ this straightforward signaling behavior by replacing the extracellular binding domain, which in wild-type Notch contains multiple EGF-like repeats, with an antibody derivative, and replacing the cytoplasmic domain with a transcription activator of choice, but still relying on the Notch NRR (L. Morsut et al., Cell (2016) 164:780-91). Generally, SynNotch signaling correlates with ligand binding, but it is difficult to adjust the sensitivity and response of the receptor. Additionally, the NRR spans approximately 160 amino acids, making this domain alone the size of some mature proteins, such as insulin or epidermal growth factor (EGF). This makes expression of the SynNotch receptor less efficient, and in some cases, the size of the entire synthetic construct can exceed the capacity of some cloning and transfection vectors.

ROBO (Roundabout) receptors are another class of cell surface receptors, which like Notch are highly conserved throughout the animal kingdom. Robo, in a manner similar to Notch, releases a nuclear transcription factor domain following ligand-induced cleavage of the extracellular portion of the receptor by ADAM10 and gamma secretase (H. Blockus et al., Development (2016) 143:3037-44). Despite this superficial functional similarity, however, ROBO does not contain a LIN/Notch domain, EGF-like repeats, or a heterodimerization domain. Additionally, the primary ligand for ROBO is a soluble protein (SLIT). Mammals have four ROBO receptors: ROBO1-3 have five immunoglobulin-like (Ig) domains, three fibronectin (Fn) repeats, and a transmembrane domain linked to an intracellular domain. ROBO4 has only two Ig domains and two Fn domains. ROBO receptors have not previously been employed in the construction of synthetic receptors. The disclosure herein provides solutions to the problems discussed herein and provides additional advantages as well.

### SUMMARY

The present disclosure provides synthetic chimeric receptors that, surprisingly, function despite the replacement of the Notch extracellular domain, including the negative regulatory region, with a portion of the ROBO1 extracellular domain, including one or more of the fibronectin (Fn) repeats. These receptors provide a range of sensitivity, including a receptor that is sensitive to the degree of T cell activation when it is expressed in an activated T cell.

In one aspect, provided herein are chimeric polynucleotides including, from N-terminus to C-terminus: (a) an extracellular ligand binding domain (ECD) having a binding affinity for a selected ligand; (b) a portion of a ROBO juxtamembrane domain (JMD) including an Fn repeat; (c) a transmembrane domain (TMD) including one or more ligand-inducible proteolytic cleavage sites; and (d) an intracellular domain (ICD) including a transcription regulator, wherein binding of the selected ligand to the extracellular binding domain induces cleavage at the ligand-inducible proteolytic cleavage site between the transcription regulator and the linking polypeptide. In some embodiments, the chimeric polypeptide does not include a LIN-12-Notch repeat (LNR) and/or a heterodimerization domain (HD) of a Notch receptor. In an embodiment, the chimeric receptor does not include a Notch NRR.

In some embodiments, the linking polypeptide includes one, two, or three Robo Fn repeats, and a short sequence of from about two to about 20 amino acids. In some embodiments, the short sequence has a degree of sequence identity with the corresponding portion of the Robo1 JMD, between the TMD and the Fn repeat domain. In some embodiments, the short sequence has a degree of sequence identity with the corresponding portion of the Notch JMD, between the TMD and the NRR domain. In some embodiments, the short sequence has less than about 60% sequence identity with the Robo1 JMD or the Notch JMD. In an embodiment, the linking polypeptide includes three or fewer Fn repeats. In an embodiment, the linking polypeptide includes two or fewer Fn repeats. In an embodiment, the linking polypeptide includes no more than one Fn repeat.

In some embodiments, the ECD includes an antigen-binding moiety capable of binding to a ligand on the surface of a cell. In some embodiments, the cell is a pathogen. In some embodiments, the ligand includes a protein or a carbohydrate. In some embodiments, the ligand is a cluster of differentiation (CD) marker. In some embodiments, the CD marker is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, BCMA (CD269), ALPPL2, GFP, eGFP, and SIRPa.

In another aspect, provided herein are nucleic acids including a nucleotide sequence encoding a chimeric polypeptide as disclosed herein. In some embodiments, the nucleotide sequence is incorporated into an expression cassette or an expression vector.

In another aspect, provided herein are recombinant cells including (a) a chimeric polypeptide as disclosed herein and/or (b) a recombinant nucleic acid as disclosed herein. In another aspect, further provided herein are cell cultures including at least one recombinant cell as disclosed herein and a culture medium.

In another aspect, provided herein are pharmaceutical compositions including a pharmaceutically acceptable carrier and one or more of the following: (a) a recombinant nucleic acid as disclosed herein, or (b) a recombinant cell as disclosed herein. In some embodiments, the disclosed pharmaceutical composition includes a recombinant nucleic acid as disclosed herein and a pharmaceutically acceptable carrier. In some embodiments, the recombinant nucleic acid is encapsulated in a viral capsid or a lipid nanoparticle.

In another aspect, provided herein are methods for modulating an activity of a cell, including: (a) providing a recombinant cell of the disclosure, and (b) contacting it with a selected ligand, wherein binding of the selected ligand to the extracellular binding domain induces cleavage of a ligand-inducible proteolytic cleavage site and releases the transcriptional regulator, wherein the released transcriptional regulator modulates an activity of the recombinant cell. Another aspect relates to methods for inhibiting an activity of a target cell in an individual, including administering to the individual an effective number of the recombinant cell of the disclosure, wherein the recombinant cell inhibits an activity of the target cell in the individual.

In another aspect, provided herein are methods for treating a health condition (e.g., disease) in an individual, the methods including a step of administering to the individual an effective number of the recombinant cell of the disclosure, wherein the recombinant cell treats the health condition in the individual.

In another aspect, provided herein are systems for modulating an activity of a cell, modulating an activity of a target cell, or treating a health condition (e.g., disease) in an individual in need thereof, wherein the system includes one or more of: a chimeric polypeptide of the disclosure; a polynucleotide of the disclosure; a recombinant cell of the disclosure; or a pharmaceutical composition of the disclosure.

In another aspect, provided herein are methods for making a recombinant cell of the disclosure, including: (a) providing a cell capable of protein expression; and (b) contacting the provided cell with a recombinant nucleic acid of the disclosure. In some embodiments, the cell is obtained by leukapheresis performed on a sample obtained from a human subject or patient, and the cell is contacted ex vivo. In some embodiments, the recombinant nucleic acid is encapsulated in a viral capsid or a lipid nanoparticle.

In another aspect, provided herein is the use of one or more of: a chimeric polypeptide of the disclosure, a polynucleotide of the disclosure, a recombinant cell of the disclosure, or a pharmaceutical composition of the disclosure, for the treatment of a health condition (e.g., disease). In some embodiments, the health condition is a disease (e.g., cancer).

In another aspect, provided herein is the use of one or more of: a chimeric polypeptide of the disclosure, a polynucleotide of the disclosure, a recombinant cell of the disclosure, or a pharmaceutical composition of the disclosure, in the manufacture of a medicament for the treatment of a health condition.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1B schematically illustrate differences between a SynNotch receptor and a chimeric polypeptide of the disclosure. FIG. 1A depicts the schematic structure of an existing first-generation synthetic Notch (SynNotch) receptor, having a juxtamembrane region that includes the Notch regulatory region. FIG. 1B depicts the schematic structure of an exemplary second-generation synthetic Notch receptor as disclosed herein (Fn Notch receptor). In this exemplary Fn Notch receptor, the Notch juxtamembrane region has been deleted and replaced with a truncated Robo1 juxtamembrane region. In each of these receptors, the extracellular binding domain contains a single-chain antigen-binding fragment (scFv) having a binding affinity for a selected ligand, which in this example is B-lymphocyte antigen CD19.

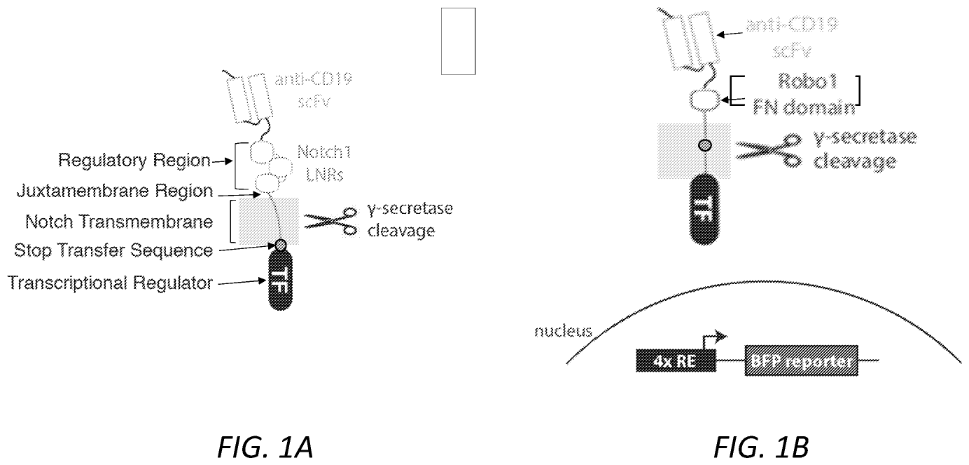

FIGS. 2A-2C schematically summarize the results of experiments performed regarding the Fn Notch receptor described herein. FIG. 2A schematically depicts (left) a first generation SynNotch receptor, (middle) an Fn Notch receptor having a Robo1 Fn domain instead of the Notch NRR, with the Fn domain linked to the TMD with a polypeptide from Notch1 (lacking the NRR), and (right) an Fn Notch receptor having a Robo1 Fn domain instead of the Notch sequence, including Robo1 sequence between the Fn domain and the TMD. FIG. 2B depicts flow cytometry data of receptor expression obtained with the corresponding receptors described in FIG. 2A. Primary human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive mCitrine gene located on the reporter plasmid. Double positive cells were sorted for on Day 5 post initial T cell stimulation and expanded further for activation testing. FIG. 2C shows the results of receptor activation testing without TCR activation. 1×10⁵ double positive T cells expressing anti-CD19 receptors were co-cultured with: nothing (upper trace), 1×10⁵ K562 cells (middle trace) or 1×10⁵ CD19+ K562 cells (lower trace) for 24 hours with each corresponding receptor. Transcriptional activation of an inducible BFP reporter gene was measured using a Fortessa X-50 (BD Biosciences).

FIG. 3 schematically summarizes the results of receptor activation testing of the receptors depicted in FIG. 2A, with concurrent T cell activation. To trigger T cell activation, anti-MCAM, anti-CD3 Bi-specific T cell Engagers (MCAM BiTE®s) were used, which activate the T cell receptor in the presence of (MCAM+) K562 cells. 1×10⁵ double positive T cells expressing anti-CD19 receptors were co-cultured with: MCAM BiTEs (upper trace), 1×10⁵ K562 cells+MCAM BiTEs (middle trace), or 1×10⁵ CD19+ K562 cells+MCAM BiTEs (lower trace) for 24 hours. Transcriptional activation of an inducible BFP reporter gene was measured using a Fortessa X-50 (BD Biociences).

FIG. 4A schematically depicts Fn Notch receptors having synthetic linker substitutions in the linking polypeptide. Exemplified here as linking polypeptides are: Robo1 sequence, (GGS)₃, (GSS)₂, (GSS)₁, and none (a direct bond). FIG. 4B shows the flow cytometry data of receptor expression for each variation. Primary human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive fluorescent protein located on the reporter plasmid. Double positive cells were sorted for on Day 5 post initial T cell stimulation and expanded further for activation testing.

FIG. 5A shows receptor activation testing without TCR activation. 1×10⁵ double positive T cells expressing anti-CD19 receptors were co-cultured with: nothing (upper trace), 1×10⁵ K562 cells (middle trace) or 1×10⁵ CD19+ K562 cells (lower trace) for 24 hours. Transcriptional activation of an inducible BFP reporter gene was measured using a Fortessa X-50 (BD Biosciences). FIG. 5B shows receptor activation with TCR activation. Phorbol 12-myristate 13-acetate (PMA), a diacyl glycerol analog, was added to all cultures and co-cultures to trigger PKC signaling.

FIG. 6 illustrates Fn Notch receptors having different ligand binding domains. FIG. 6A shows Fn Notch expression testing with an anti-GFP LagG17 nanobody or anti-ALPPL2 scFv ligand binding domains. Primary CD4 human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive fluorescent protein found on the reporter plasmid. Double positive cells were sorted for on Day 5 post initial T cell stimulation and expanded further for activation testing. FIG. 6B shows flow cytometry data for receptor activation. 1×10⁵ double positive CD8+ T cells expressing anti-GFP or anti-ALPPL2 Fn Notch were co-cultured with: nothing (upper trace), 1×10⁵ K562 cells (middle trace), or 1×10⁵ surface GFP K562 cells/ALPPL2+ K562 cells (lower trace) for 24 hours. Transcriptional activation of an inducible BFP reporter gene was subsequently measured using a Fortessa X-50 (BD Biosciences).

### DETAILED DESCRIPTION OF THE DISCLOSURE

The present disclosure generally relates to, among other things, a new class of engineered chimeric polypeptide receptors, which modulate transcriptional regulation in a ligand-dependent manner. Particularly, the new receptors (termed “Fn Notch”), even though derived from Notch, do not require the Notch negative regulatory regions (NRR) previously believed to be essential for synthetic receptor function. The Fn Notch receptors of the disclosure contain one or more fibronectin (“Fn”) repeats from the Robo1 receptor, but function without the need for any further Robo1 sequence or regulatory feature. These receptors are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. The disclosure also provides compositions and methods useful for producing such receptors, nucleic acids encoding same, host cells genetically modified with these nucleic acids, as well as methods for modulating an activity of a cell and/or for the treatment of various health conditions, such as diseases (e.g., cancers).

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a composition or formulation by an administration route including, but not limited to, intravenous, intracerebral, intrathecal, intra-arterial, intramuscular, intraperitoneal, subcutaneous, intramuscular, and combinations thereof. The term includes, but is not limited to, administration by a medical professional and self-administration.

“Cancer” refers to the presence of cells possessing characteristics typical of cancer-causing cells, such as uncontrolled proliferation, immortality, metastatic potential, rapid growth and proliferation rate, and certain characteristic morphological features. Some types of cancer cells can aggregate into a mass, such as a tumor, but some cancer cells can exist alone within a subject. A tumor can be a solid tumor, a soft tissue tumor, or a metastatic lesion. As used herein, the term “cancer” also encompass other types of non-tumor cancers. Non-limiting examples include blood cancers or hematological malignancies, such as leukemia, lymphoma, and myeloma. Cancers can include premalignant, as well as malignant cancers.

The terms “host cell” and “recombinant cell” are used interchangeably herein. It is understood that such terms, as well as “cell”, “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the originally cell or cell line.

The term “operably linked”,” as used herein, denotes a physical or functional linkage between two or more elements, e.g., polypeptide sequences or polynucleotide sequences, which permits them to operate in their intended fashion.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, and unless otherwise specified, a “therapeutically effective amount” of an agent is an amount sufficient to provide a therapeutic benefit in the treatment or management of a health condition, such as a disease (e.g., a cancer), or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” is an individual under the care of a physician. Thus, the subject can be a human individual or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and the like. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

One skilled in the art will understand that the chimeric polypeptide receptors disclosed herein facilitate amplified activation under certain cellular and environmental contexts. This type of feedback on the receptor activity is a new feature that can be exploited to enhance and tune the production of therapeutic payloads by engineered cells. Furthermore, as described in greater detail below, a number of the receptor variants disclosed herein are easier to express than existing SynNotch receptors, possibly due to their smaller size.

### Notch Receptors

Notch receptors are large transmembrane proteins that normally communicate signals upon binding to surface-bound ligands expressed on adjacent cells. Notch signals rely on cell-cell contact. Evolutionary divergence of vertebrates and invertebrates has been accompanied by at least two rounds of gene duplication: flies possess a single Notch gene, worms two (GLP-1 and LIN-12), and mammals four (NOTCH1-4). Transduction of Notch signals relies on three key events: (i) ligand recognition, (ii) conformational exposure of the ligand-dependent cleavage site, and (iii) assembly of nuclear transcriptional activation complexes.

Canonical Notch signals are transduced by a process called regulated intramembrane proteolysis. Notch receptors are normally maintained in a resting, proteolytically resistant conformation on the cell surface, but ligand binding initiates a proteolytic cascade that releases the intracellular portion of the receptor (ICD) from the membrane. The critical, regulated cleavage step is effected using ADAM metalloproteases and occurs at a site called S2 immediately external to the plasma membrane. This truncated receptor, dubbed NEXT (for Notch extracellular truncation), remains membrane tethered until it is processed at site S3 by gamma secretase, a multiprotein enzyme complex.

After gamma secretase cleavage, the ICD ultimately enters the nucleus, where it assembles a transcriptional activation complex that contains a DNA-binding transcription factor called CSL, and a transcriptional coactivator of the Mastermind family. This complex then engages additional coactivator proteins such as p300 to recruit the basal transcription machinery and activate the expression of downstream target genes.

Notch receptors have a modular domain organization. The ectodomains of Notch receptors consist of a series of N-terminal epidermal growth factor receptor (EGF)-like repeats that are responsible for ligand binding. O-linked glycosylation of these EGF-like repeats, including modification by 0-fucose, Fringe, and Rumi glycosyltransferases, also modulates the activity of Notch receptors in response to different ligand subtypes in flies and mammals.

The EGF-like repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular, the receptor has a transmembrane segment and an intracellular domain (ICD), which includes a transcriptional regulator.

### Roundabout Receptor 1 (Robo1)

ROBO1 belongs to the Roundabout receptor family, which are single-pass type I membrane proteins that belong to the immunoglobulin (Ig) superfamily of cell adhesion molecules (CAMs). Robo receptors are evolutionarily conserved across bilateral anatomical species. Three Robo receptors (Robo, Robo2, and Robo3) have been characterized in Drosophila, Zebrafish, and chicken while C. elegans contains a single robo ortholog, SAX-3. Four Robo receptors (Robo1-4) have been identified in vertebrates. The Drosophila and vertebrate Robo1-3 are most similar, containing five immunoglobulin (Ig) and three fibronectin (Fn) domains in their extracellular region. Robo4 is a smaller endothelial and vascular specific receptor, having only two Ig and Fn domains. These extracellular domains are followed by a membrane proximal region, a single transmembrane helix, and an unstructured intracellular region containing conserved sequence motifs used to mediate the binding of effector proteins. The crystal structures of several extracellular domains of Robo1 have been determined, these include the Ig1-2 region harboring the Slit2 ligand binding region on Ig1, and the juxtamembrane region spanning Fn2-3.

The human homolog of the Drosophila roundabout (robo) gene, ROBO1, encodes an axon guidance receptor. The main function of ROBO1 is to interact with Slit Guidance Ligand (SLIT) as an axon guidance receptor. The Slit-ROBO1 interaction was firstly described that transduces signals modifying repulsive cues on axons and growth cones in neural development and regulates chemotaxis of T cells and monocytes. ROBO1 contains five repeats of immunoglobulin (Ig) domains, three repeats of fibronectin Type-III (Fn) domains, a transmembrane domain, and an intracellular tail. Among these domains, the structure of the first Ig domain has been determined by X-ray crystallographic analysis as the complex with the second leucine-rich repeat domain of SLIT2, a known ligand for ROBO1. The third Fn domain of ROBO1 (Fn3) is located closest to the transmembrane region.

Robo1 Fn domains are estimated to be present in about 2% of all human proteins and found in organisms as evolutionarily distant as bacteriophages. Moreover, Fn domain has a stable framework structure and consequently a high thermostability, which is utilized as a scaffold for the generation of stable proteins in the protein engineering. Therefore, in ROBO1, Fn domains have been reported to contribute to stabilizing the extracellular region and the interaction with SLIT2.

### Compositions of the Disclosure

The receptors of the disclosure provide a range of sensitivity, including a receptor that is sensitive to the degree of T cell activation when it is expressed in a T cell. Additionally, by omitting the Notch regulatory regions, polynucleotides encoding the receptors of the disclosure can be made smaller than SynNotch-encoding polynucleotides, which enables the use of vectors having more limited capacity, or the inclusion of additional elements that would otherwise be excluded by vector capacity-related size constraints.

As described in greater detail below, several chimeric polypeptide receptors disclosed herein have better activity than existing SynNotch receptors and provide a more modular platform for engineering. Existing SynNotch receptors can be engineered with ligand-binding domains such scFvs and nanobodies, but it has been difficult to use natural extracellular domains from receptors/ligands on SynNotch receptors. In contrast, a number of the second-generation Notch receptors disclosed herein are amenable to use with other types of ligand binding domains, thus expanding the landscape of targetable diseases and tissues.

As described herein, chimeric polypeptide receptors have been tested and validated in primary human T cells. Without being bound to any particular theory, it is contemplated that these new receptors show similar performance in mouse models. The receptors disclosed herein may be engineered into various immune cell types for enhanced discrimination and elimination of tumors, or into other cell types for control of autoimmunity and tissue regeneration. Accordingly, engineered cells, such as immune cells engineered to express one of more of the chimeric receptors disclosed herein, are also within the scope of the disclosure.

### Chimeric Polypeptides

This disclosure provides novel, non-naturally occurring recombinant chimeric receptors engineered to modulate transcriptional regulation in a ligand-dependent manner. Particularly, the new receptors, even though derived from Notch, and containing elements of Robo, do not require either Robo regulatory regions or the Notch regulatory regions previously believed to be necessary for the functioning of the receptors. In some embodiments, the receptors disclosed herein bind a target cell-surface displayed ligand, which triggers proteolytic cleavage of the receptors and release of a transcriptional regulator that modulates a custom transcriptional program in the cell.

In some embodiments, provided herein is a chimeric polypeptide including, from N-terminus to C-terminus: (a) an extracellular ligand binding domain (ECD) having a binding affinity for a selected ligand; (b) a linking polypeptide (in Notch, this would correspond to the JMD, juxtamembrane domain); (c) a transmembrane domain (TMD) including one or more ligand-inducible proteolytic cleavage sites; and (d) an intracellular domain (ICD) including a transcription regulator, wherein binding of the selected ligand to the extracellular binding domain induces cleavage at the ligand-inducible proteolytic cleavage site between the transcription regulator and the linking polypeptide. In some embodiments, the chimeric polypeptide of the disclosure does not include an NRR, LNR, and/or an HD of a Notch receptor. In some embodiments, the chimeric receptor does not include an LNR. In some embodiments, the chimeric polypeptide does not include an HD of a Notch receptor. In some embodiments, the chimeric polypeptide does not include an NRR of a Notch receptor.

### Extracellular Domains (ECD)

In some embodiments, the ECD of the chimeric receptors disclosed herein has a binding affinity for one or more target ligands. The target ligand is expressed on a cell surface, or is otherwise immobilized or restrained so that it can exert a mechanical force on the chimeric receptor. For example, an otherwise soluble ligand may be targeted if it is bound to a surface, or to a molecule in the extracellular matrix. In some embodiments, the target ligand is a cell-surface ligand. Non-limiting examples of suitable ligands include cell surface receptors; adhesion proteins; carbohydrates, lipids, glycolipids, lipoproteins, and lipopolysaccharides that are surface-bound; integrins; mucins; and lectins. In some embodiments, the ligand is a protein. In some embodiments, the ligand is a carbohydrate.

In some embodiments, the ligand is a cluster of differentiation (CD) marker. In some embodiments, the CD marker is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE.

In some embodiments, the ECD includes the ligand-binding portion of a receptor. In some embodiments, the ECD includes an antigen-binding moiety that binds to one or more target antigens. In some embodiments, the antigen-binding moiety includes one or more antigen-binding determinants of an antibody or a functional antigen-binding fragment thereof. In some embodiments, the antigen-binding moiety is selected from the group consisting of an antibody, a nanobody, a diabody, a triabody, or a minibody, a F(ab′)2 fragment, a Fab fragment, a single chain variable fragment (scFv), and a single domain antibody (sdAb), or a functional fragment thereof. In some embodiments, the antigen-binding moiety includes an scFv.

The antigen-binding moiety can include naturally-occurring amino acid sequences or can be engineered, designed, or modified so as to provide desired and/or improved properties, e.g., binding affinity. Generally, the binding affinity of an antigen-binding moiety, e.g., an antibody, for a target antigen (e.g., CD19 antigen) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding moiety that binds the antigen with high affinity and does not significantly bind other unrelated antigens.

A skilled artisan can select an ECD based on the desired localization or function of a cell that is genetically modified to express chimeric polypeptide or Fn Notch receptor of the present disclosure. For example, the ECD can target cells to estrogen-dependent breast cancer cells. In some embodiments, the ECD of the disclosed chimeric polypeptide Fn Notch receptors is capable of binding a tumor-associated antigen (TAA) or a tumor-specific antigen (TSA). A skilled artisan in the art will understand that TAAs include a molecule, such as e.g., a protein, present on tumor cells and on normal cells, or on many normal cells, but at much lower concentration than on tumor cells. In contrast, TSAs generally include a molecule, such as e.g., a protein which is present on tumor cells but absent from normal cells.

In some cases, the antigen-binding moiety is specific for an epitope present in an antigen that is expressed by a tumor cell, i.e., a tumor-associated antigen. The tumor cell associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a colorectal cancer cell, and the like. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

Non-limiting examples of suitable target antigens include CD19, B7H3 (CD276), BCMA (CD269), alkaline phosphatase, placental-like 2 (ALPPL2), green fluorescent protein (GFP), enhanced green fluorescent protein (EGFP), signal regulatory protein α (SIRPα), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-beta, PRSS21, PSCA, PSMA, ROR1, SSEA-4, TAG72, TEM1/CD248, TEM7R, TSHR, VEGFR2, ALPI, citrullinated vimentin, cMet, and Axl.

In some embodiments, the target antigen is selected from CD19, B7H3 (CD276), BCMA (CD269), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-beta, PRSS21, PSCA, PSMA, ROR1, SSEA-4, TAG72, TEM1/CD248, TEM7R, TSHR, VEGFR2, ALPI, citrullinated vimentin, cMet, Axl, GPC2, human epidermal growth factor receptor 2 (Her2/neu), CD276 (B7-H3), IL-13Rα1, IL-13Rα2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, LFA-1 (CD11a), myostatin, OX-40, scleroscin, SOST, TGFβ1, TNF-α, VEGF-A, pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin.

Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), Ral-B, GPC2, CD276 (B7-H3), or IL-13Rα. In some embodiments, the antigen includes ALPPL2. In some embodiments, the antigen includes BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen includes signal regulatory protein α (SIRPα).

Additional antigens that can be suitable for the chimeric polypeptide receptors disclosed herein include, but are not limited to GPC2, human epidermal growth factor receptor 2 (Her2/neu), CD276 (B7-H3), IL-13Rα1, IL-13Rα2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional suitable antigens include, but are not limited to, those associated with an inflammatory disease such as, AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, LFA-1 (CD11a), myostatin, OX-40, scleroscin, SOST, TGF beta 1, TNF-α, and VEGF-A.

Further antigens suitable for the chimeric receptors disclosed herein include, but are not limited to the pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin.

Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin (33 (CD61), galactin, K-Ras (V-Ki-ras2 Kirsten rat sarcoma viral oncogene), and Ral-B. In some embodiments, the antigen is GPC2, CD19, Her2/neu, CD276 (B7-H3), IL-13Rα1, or IL-13Rα2. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

In some embodiments, antigens suitable for targeting by the chimeric polypeptides and Fn Notch receptors disclosed herein include ligands derived from a pathogen. For example, the antigen can be HER2 produced by HER2-positive breast cancer cells. In some embodiments, the antigen can be CD19 that is expressed on B-cell leukemia. In some embodiments, the antigen can be EGFR that is expressed on glioblastoma multiform (GBM) but much less expressed so on healthy CNS tissue. In some embodiments, the antigen can be CEA that is associated with cancer in adults, for example colon cancer.

In some embodiments, the antigen-binding moiety of the ECD is specific for a cell surface target, where non-limiting examples of cell surface targets include CD19, CD30, Her2, CD22, ENPP3, EGFR, CD20, CD52, CD11a, and alpha-integrin. In some embodiments, the chimeric receptors disclosed herein include an ECD having an antigen-binding moiety that binds CD19, CEA, HER2, MUC1, CD20, or EGFR. In some embodiments, the chimeric receptors disclosed herein include an ECD including an antigen-binding moiety that binds CD19.

In some embodiments, the antigen-binding moiety includes an amino acid sequence having at least 70%, at least 75%, at least 80%, at least 85%, at least 90%, at least 95%, at least 96%, at least 97%, at least 98%, at least 99% sequence identity to one or more of SEQ ID NOS: 7-8 in the Sequence Listing. In some embodiments, the antigen-binding moiety includes an amino acid sequence having at least 90% sequence identity to a sequence selected from the group consisting of SEQ ID NOS: 7-8. In some embodiments, the antigen-binding moiety includes an amino acid sequence having at least 95% sequence identity to a sequence selected from the group consisting of SEQ ID NOS: 7-8. In some embodiments, the antigen-binding moiety includes an amino acid sequence having 100% sequence identity to one or more of SEQ ID NOS: 7-8. In some embodiments, the antigen-binding moiety includes an amino acid sequence having a sequence selected from the group consisting of SEQ ID NOS: 7-8, wherein one, two, three, four, or five of the amino acid residues in any one of the SEQ ID NOS: 7-8 is/are substituted by a different amino acid residue.

### Linking Sequence

The ECD and the TMD are linked to each other with a linking polypeptide (LP) derived from the Robo1 juxtamembrane domain with fibronectin repeats (Fn), with a short polypeptide sequence between the Fn repeats and the TMD. The linking polypeptide does not contain one or both of: the Notch negative regulatory region, or the HD domain. The linking polypeptide can contain 1, 2, 3, 4, or 5 Fn repeats. In some embodiments of the disclosure, the chimeric receptor includes a linking polypeptide having about 1 to about 5 Fn repeats, about 1 to about 3 Fn repeats, or about 2 to about 3 Fn repeats.

The short polypeptide sequence between the Fn repeats and the TMD can be from about 2 to about 30 amino acid residues (e.g., 2, 3, 4, 5, 6, 7, 8, 9, 10, 11, 12, 13, 14, 15, 16, 17, 18, 19, 20, and so forth, amino acid residues). In some embodiments, the short polypeptide sequence can be between about 5 and about 20 amino acids, of any sequence. In some embodiments, the short polypeptide sequence can be between about 5 and about 20 naturally-occurring amino acids, of any sequence. In some embodiments, the short polypeptide sequence can be between about 5 and about 20 amino acids, of any sequence but excluding proline. In some embodiments, the short polypeptide sequence can be between about 5 and about 20 amino acids, and about 50% or more of the amino acids are glycine. In some embodiments, the short sequence is a Gly-Ser polymer, such as, for example without limitation, a (GGS)ₙ polymer, where n is an integer from 1 to 50, for example, from 1 to 10, from 5 to 15, from 10 to 20, from 15 to 25, from 20 to 30, from 25 to 35, from 30 to 40, from 35 to 45, or from 40 to 50. In some embodiments, a linking polypeptide the amino acid sequence (GGS)ₙ wherein n is an integer from 1 to 10. In some embodiments, a linking polypeptide the amino acid sequence (GGS)ₙ wherein n is an integer from 10 to 20. In some embodiments, a linking polypeptide the amino acid sequence (GGS)ₙ wherein n is an integer from 20 to 30. In some embodiments, a linking polypeptide the amino acid sequence (GGS)ₙ wherein n is an integer from 30 to 40. In some embodiments, a linking polypeptide the amino acid sequence (GGS)ₙ wherein n is an integer from 40 to 50. In some embodiments, the short sequence is a (GGS)₁, (GGS)₂, (GGS)₃, (GGS)₆, (GGS)₉, (GGS)₁₂, (GGS)₁₅, or (GGS)₁₈ polymer. In some embodiments, the short sequence is, for example without limitation, a (SGG)ₙ, (GSG)ₙ, (GGGS)ₙ(SEQ ID NO: 30), (SGGG)ₙ (SEQ ID NO: 31), or a (GGXS)ₙ (SEQ ID NO: 32) polymer, where n is an integer from 1 to 50, and X is any amino acid.

In some embodiments, the short polypeptide sequence can be between about 5 and about 20 amino acids, where the amino acids are selected from glycine, serine, threonine, and alanine. In some embodiments, the short sequence has at least about 80% sequence identity to the corresponding sequence of the Robo1 JMD (i.e., the portion of the Robo1 receptor between the TMD and the most C-terminal Fn repeat). In some embodiments, the short sequence has at least about 85%, 90%, 95%, 98%, 99%, or about 100% sequence identity to the Robo1 JMD. In some embodiments, the short sequence has at least about 80% sequence identity to the corresponding sequence of the Notch1, Notch2, Notch3, or Notch4 JMD (e.g., the portion of the Notch receptor between the TMD and the NRR). In some embodiments, the short sequence has at least about 85%, 90%, 95%, 98%, 99%, or about 100% sequence identity to the Robo1, Notch1, Notch2, Notch3, or Notch4 JMD. In some embodiments, the short sequence has less than about 80% sequence identity to the Robo1, Notch1, Notch2, Notch3, or Notch4 JMD.

In some embodiments, the length and amino acid composition of the linking polypeptide can be varied to alter the orientation and/or proximity of the ECD and the TMD relative to one another to achieve a desired activity of the chimeric polypeptide of the disclosure. In some embodiments, the linking polypeptide includes a sequence having at least 80% sequence identity, such as, at least 80%, at least 85%, at least 90%, at least 95%, at least 96%, at least 97%, at least 98%, or 99% sequence identity to a sequence selected from the group consisting of SEQ ID NOS: 9-11 and 19-20 in the Sequence Listing. In some embodiments, the linking polypeptide includes an amino acid sequence having at least 90% sequence identity to a sequence selected from the group consisting of SEQ ID NOS: 9-11 and 19-20. In some embodiments, the linking polypeptide includes an amino acid sequence having at least 95% sequence identity to a sequence selected from the group consisting of SEQ ID NOS: 9-11 and 19-20. In some embodiments, the linking polypeptide includes an amino acid sequence having at least 100% sequence identity to a sequence selected from the group consisting of SEQ ID NOS: 9-11 and 19-20. In some embodiments, the linking polypeptide includes an amino acid sequence having a sequence selected from the group consisting of SEQ ID NOS: 9-11 and 19-20, wherein one, two, three, four, or five of the amino acid residues in any one of the SEQ ID NOS: 9-11 and 19-20 is/are substituted by a different amino acid residue.

### Transmembrane Domains

As described above, the chimeric polypeptides of the disclosure include a transmembrane domain including one or more ligand-inducible proteolytic cleavage sites.

Examples of proteolytic cleavage sites in a Notch receptor (e.g., S2 or S3) are as described above. Additional proteolytic cleavage sites suitable for the compositions and methods disclosed herein include, but are not limited to, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue) (SEQ ID NO: 22), e.g., Pro-X-X-Hy-(Ser/Thr) (SEQ ID NOs: 23), e.g., Pro-Leu/Gln-Gly-Met-Thr-Ser (SEQ ID NO: 24) or Pro-Leu/Gln-Gly-Met-Thr (SEQ ID NO: 25). Another example of a suitable protease cleavage site is a plasminogen activator cleavage site, e.g., a urokinase-type plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Val-Gly-Arg (SEQ ID NO: 26). Another example of a protease cleavage site that can be included in a proteolytically cleavable linker is a tobacco etch virus (TEV) protease cleavage site, e.g., Glu-Asn-Leu-Tyr-Thr-Gln-Ser (SEQ ID NO: 27), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 28), where cleavage occurs after the lysine residue. Another example of a protease cleavage site that can be included in a proteolytically cleavable linker is a thrombin cleavage site, e.g., Leu-Val-Pro-Arg (SEQ ID NO: 29). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr virus protease, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermolysin, fibroblast collagenase and stromelysin-1, matrix metalloproteinase 13 (collagenase-3), tissue-type plasminogen activator(tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the Fn Notch is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

Generally, the transmembrane domain (TMD) suitable for the chimeric receptors disclosed herein can be any transmembrane domain of a Type 1 transmembrane receptor including at least one γ-secretase cleavage site. Detailed description of the structure and function of the γ-secretase complex as well as its substrate proteins, including amyloid precursor protein (APP) and Notch, can, for example, be found in a recent review by Zhang et al., Frontiers Cell Neurosci (2014). Non-limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one γ-secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD known for Notch receptors. In some embodiments, the TMD of the chimeric polypeptides or Notch receptors of the disclosure is a TMD derived from a different Notch receptor. For example, in an Fn Notch based on human Notch1, the Notch1 TMD can be substituted with a Notch3 TMD, a Notch4 TMD, or a Notch TMD from a non-human animal such as Danio rerio, Drosophila melanogaster, Xenopus laevis, or Gallus gallus.

In some embodiments, the transmembrane domain includes an amino acid sequence exhibiting at least 70%, at least 75%, at least 80%, at least 85%, at least 90%, at least 95%, at least 96%, at least 97%, at least 98%, at least 99% sequence identity to one or more of SEQ ID NOS: 12-13 and 21 in the Sequence Listing. In some embodiments, the transmembrane domain includes an amino acid sequence having at least 90% sequence identity to a sequence selected from the group consisting of SEQ ID NOS: 12-13 and 21. In some embodiments, the transmembrane domain includes an amino acid sequence having at least 95% sequence identity to a sequence selected from the group consisting of SEQ ID NOS: 12-13 and 21. In some embodiments, the transmembrane domain includes an amino acid sequence having 100% sequence identity to one or more of SEQ ID NOS: 12-13 and 21. In some embodiments, the transmembrane domain includes an amino acid sequence having a sequence selected from the group consisting of SEQ ID NOS: 12-13 and 21, wherein one, two, three, four, or five of the amino acid residues in any one of the SEQ ID NOS: 12-13 and 21 is/are substituted by a different amino acid residue.

In some embodiments, the amino acid substitution(s) within the TMD includes one or more substitutions within a “GV” motif of the TMD. In some embodiments, at least of such substitution(s) is a substitution to alanine. For example, one, two, three, four, five, or more of the amino acid residues of the sequence FMYVAAAAFVLLFFVGCGVLLS (SEQ ID NO: 13) may be substituted by a different amino acid residue. In some embodiments, the amino acid residue at position 18 and/or 19 of the “GV” motif within SEQ ID NO: 13 is substituted by a different amino acid residue. In some embodiments, the glycine residue at position 18 of SEQ ID NO: 13 is substituted by a different amino acid residue. In some embodiments, the valine residue at position 19 of SEQ ID NO: 13 is substituted by a different amino acid residue. In some embodiments, the transmembrane domain includes an amino acid sequence having a sequence corresponding to SEQ ID NO: 13 with a mutation at the position corresponding to position 18 of SEQ ID NO: 13, such as G19A mutations. In some embodiments, the transmembrane domain includes an amino acid sequence having a sequence corresponding to SEQ ID NO: 28 with a mutation at the position corresponding to position 19 of SEQ ID NO: 13, such as V19A mutations.

### Stop-Transfer Sequences

In some embodiments, the chimeric receptors of the disclosure include a stop-transfer sequence (STS) which consists of a highly-charged domain located between the TMD and the ICD. Without being bound to any particular theory, such a highly-charged domain disposed between the TMD and the ICD prevents the ICD from entering the membrane. In principle, there are no particular limitations to the length and/or amino acid composition of the STS. In some embodiments, any arbitrary single-chain peptide including about 1 to about 40 amino acid residues (e.g., 1, 2, 3, 4, 5, 6, 7, 8, 9, 10, 11, 12, 13, 14, 15, 16, 17, 18, 19, or 20, and so forth, amino acid residues) can be used as a STS. In some embodiments, the STS includes about 2 to 15, about 4 to 20, about 8 to 25, about 10 to 30, about 12 to 35, about 14 to 40, about 5 to 40, about 10 to 35, about 15 to 30, about 20 to 25, about 20 to 40, about 10 to 30, about 4 to 20, or about 5 to 25 amino acid residues. In some embodiments, the STS includes about 1 to 10, about 5 to 12, about 6 to 14, about 7 to 18, about 8 to 20, about 9 to 22, about 10 to 24, or about 11 to 26 amino acid residues. In some embodiments, the STS includes about 4 to 10 residues, such as, 4, 5, 6, 7, 8, 9, or 10 amino acid residues.

In some embodiments, the STS includes a sequence having at least 70% sequence identity, such as, at least 70%, at least 75%, at least 80%, at least 85%, at least 90%, at least 95%, at least 96%, at least 97%, at least 98%, or 99% sequence identity to a STS sequence from Notch1, Notch2, Notch3, Notch4, CSF1R, CXCL16, DAG1, GHR, PTPRF, AGER, KL, NRG1, LRP1B, Jag2, EPCAM, KCNE3, CDH2, NRG2, PTPRK, BTC, EPHA3, IL1R2, or PTPRM. In some embodiments, the STS includes a sequence comprising only Lys (K) or Arg (R) in the first 4 residues. In some embodiments, the STS includes one, two, three, four, five, or more basic residues. In some embodiments, the STS includes five, four, three, two, one, or zero aromatic residues or residues with hydrophobic and/or bulky side chains.

In some embodiments, the STS includes a sequence having at least 80% sequence identity, such as, at least 80%, at least 85%, at least 90%, at least 95%, at least 96%, at least 97%, at least 98%, or 99% sequence identity to SEQ ID NO: 14 in the Sequence Listing. In some embodiments, the STS includes an amino acid sequence having at least 90% sequence identity to SEQ ID NO: 14. In some embodiments, the STS includes an amino acid sequence having at least 95% sequence identity to SEQ ID NO: 14. In some embodiments, the STS includes an amino acid sequence having at least 100% sequence identity to SEQ ID NO: 14. In some embodiments, the STS includes an amino acid sequence of SEQ ID NO: 14, wherein one, two, three, four, or five of the amino acid residues in SEQ ID NO: 14 is/are substituted by a different amino acid residue.

### Intracellular Domain (ICD)

The chimeric receptor of the disclosure includes a transcriptional regulator. The transcriptional regulator of the disclosure is a biochemical element that acts to promote or inhibit the transcription of a promoter-driven DNA sequence. Transcriptional regulators suitable for the compositions and methods of the disclosure can be naturally-occurring transcriptional regulators or can be engineered, designed, or modified so as to provide desired and/or improved properties, e.g., modulating transcription. In some embodiments, the transcription regulator directly regulates differentiation of the cell. In some embodiments, the transcription regulator indirectly regulates differentiation of the cell by modulating the expression of a second transcription factor. It will be understood by one having ordinary skill in the art that a transcriptional regulator can be a transcriptional activator or a transcriptional repressor. In some embodiments, the transcriptional regulator is a transcriptional repressor. In some embodiments, the transcriptional regulator is a transcriptional activator. In some embodiments, the transcription regulator can further include a nuclear localization signal. In some embodiments, the transcription regulator is selected from Ga14-VP16, Ga14-VP64, tetR-VP64, ZFHD1-VP64, Ga14-KRAB, and HAP1-VP16. In some embodiments, the transcription regulator is Ga14-VP64.

In some embodiments, the ICD includes a sequence having at least 80% sequence identity, such as, at least 80%, at least 85%, at least 90%, at least 95%, at least 96%, at least 97%, at least 98%, or 99% sequence identity to SEQ ID NO: 15 in the Sequence Listing. In some embodiments, the ICD includes an amino acid sequence having at least 90% sequence identity to SEQ ID NO: 15. In some embodiments, the ICD includes an amino acid sequence having at least 95% sequence identity to SEQ ID NO: 15. In some embodiments, the ICD includes an amino acid sequence having at least 100% sequence identity to SEQ ID NO: 15. In some embodiments, the ICD includes an amino acid sequence of SEQ ID NO: 15, wherein one, two, three, four, or five of the amino acid residues in SEQ ID NO: 15 is/are substituted by a different amino acid residue.

### Additional Domains

In some embodiments, the Notch extracellular domains located N-terminally to the TMD can further include an additional region, for example a membrane localization signal such as a CD8A signal, a detectable marker such as a myc tag or His tag, and the like.

In some embodiments, the chimeric polypeptide of the disclosure includes: (a) a linking polypeptide including an amino acid sequence having at least 80% sequence identity to any one of SEQ ID NO: 9-11 and 19-20; (b) a transmembrane domain including an amino acid sequence having at least 80% sequence identity to any one of SEQ ID NOS: 12-13 and 21; and (c) a stop transfer sequence domain including an amino acid sequence having at least 80% sequence identity to SEQ ID NO: 14. In some embodiments, the chimeric polypeptide of the disclosure includes: (a) a linking polypeptide including an amino acid sequence having at least 90% sequence identity to any one of SEQ ID NO: 9-11 and 19-20; (b) a transmembrane domain including an amino acid sequence having at least 90% sequence identity to any one of SEQ ID NOS: 12-13 and 21; and (c) a stop transfer sequence domain including an amino acid sequence having at least 90% sequence identity to SEQ ID NO: 14. In some embodiments, the chimeric polypeptide of the disclosure includes: (a) a linking polypeptide including an amino acid sequence having at least 95% sequence identity to any one of SEQ ID NO: 9-11 and 19-20; (b) a transmembrane domain including an amino acid sequence having at least 95% sequence identity to any one of SEQ ID NOS: 12-13 and 21; and (c) a stop transfer sequence domain including an amino acid sequence having at least 95% sequence identity to SEQ ID NO: 14.

In some embodiments, the chimeric polypeptide of the disclosure includes: (a) a linking polypeptide including an amino acid sequence selected from the group consisting of SEQ ID NO: 9-11 and 19-20; (b) a transmembrane domain including an amino acid sequence selected from the group consisting of SEQ ID NOS: 12-13 and 21; and (c) a stop transfer sequence domain including an amino acid sequence selected from SEQ ID NO: 14.

In some embodiments, the chimeric polypeptide of the disclosure includes: (a) a linking polypeptide including an amino acid sequence selected from the group consisting of SEQ ID NO: 9-11 and 19-20, wherein one, two, three, four, or five of the amino acid residues in any one of the SEQ ID NOS: 9-11 and 19-20 is/are substituted by a different amino acid residue; (b) a transmembrane domain including an amino acid sequence selected from the group consisting of SEQ ID NOS: 12-13 and 21, wherein one, two, three, four, or five of the amino acid residues in any one of the SEQ ID NOS: 12-13 and 21 is/are substituted by a different amino acid residue; and (c) a stop transfer sequence domain including an amino acid sequence of SEQ ID NO: 14, wherein one, two, three, four, or five of the amino acid residues in SEQ ID NO: 14 is/are substituted by a different amino acid residue.

In some embodiments, the chimeric receptor of the disclosure includes an amino acid sequence having at least about 80%, 90%, 95%, 96%, 97, 98%, 99%, or 100% sequence identity to a chimeric receptor disclosed herein. In some embodiments, provided herein are chimeric receptors including an amino acid sequence having at least about 80%, 90%, 95%, 96%, 97, 98%, 99%, or 100% sequence identity to any one of SEQ ID NOs: 1-6 identified in the Sequence Listing. In some embodiments, the chimeric receptors include an amino acid sequence having at least about 80%, 90%, 95%, 96%, 97, 98%, 99%, or 100% sequence identity to SEQ ID NO: 1. In some embodiments, the chimeric receptors include an amino acid sequence having at least about 80%, 90%, 95%, 96%, 97, 98%, 99%, or 100% sequence identity to SEQ ID NO: 2. In some embodiments, the chimeric receptors include an amino acid sequence having at least about 80%, 90%, 95%, 96%, 97, 98%, 99%, or 100% sequence identity to SEQ ID NO: 3. In some embodiments, the chimeric receptors include an amino acid sequence having at least about 80%, 90%, 95%, 96%, 97, 98%, 99%, or 100% sequence identity to SEQ ID NO: 4. In some embodiments, the chimeric receptors include an amino acid sequence having at least about 80%, 90%, 95%, 96%, 97, 98%, 99%, or 100% sequence identity to SEQ ID NO: 5. In some embodiments, the chimeric receptors include an amino acid sequence having at least about 80%, 90%, 95%, 96%, 97, 98%, 99%, or 100% sequence identity to SEQ ID NO: 6.

### Nucleic Acid Molecules

In one aspect, some embodiments disclosed herein relate to nucleic acid molecules that include nucleotide sequences encoding the Fn Notch receptors of the disclosure, including expression cassettes, and expression vectors containing these nucleic acid molecules operably linked to heterologous nucleic acid sequences such as, for example, regulator sequences which facilitate in vivo expression of the receptor in a host cell.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence that encodes a chimeric polypeptide including, from N-terminus to C-terminus: (a) an extracellular binding domain having a binding affinity for a selected ligand; (b) a linking polypeptide; (c) a transmembrane domain including one or more ligand-inducible proteolytic cleavage sites; and (d) an intracellular domain including a transcription regulator, wherein binding of the selected ligand to the extracellular binding domain induces cleavage at the ligand-inducible proteolytic cleavage site between the transcription regulator and the linking polypeptide. In some embodiments, the chimeric polypeptide of the disclosure does not include a LIN-12-Notch repeat (LNR) and/or a heterodimerization domain (HD) of a Notch receptor.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. Thus, in some embodiments, an expression cassette of the disclosure includes a coding sequence for the chimeric polypeptide as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. Retroviral vectors used herein contain structural and functional genetic elements, or portions thereof, that are primarily derived from a retrovirus. Similarly, lentiviral vectors contain structural and functional genetic elements, or portions thereof including LTRs, that are primarily derived from a lentivirus.

In some embodiments, provided herein are nucleic acid molecules encoding a polypeptide with an amino acid sequence having at least about 80%, 90%, 95%, 96%, 97, 98%, 99%, or 100% sequence identity to a chimeric receptor disclosed herein. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOS: 1-6 identified in the Sequence Listing. In some embodiments, the nucleic acid molecules encode a polypeptide with an amino acid sequence having at least about 80%, 90%, 95%, 96%, 97, 98%, 99%, or 100% sequence identity to any one of SEQ ID NO: 1. In some embodiments, the nucleic acid molecules encode a polypeptide with an amino acid sequence having at least about 80%, 90%, 95%, 96%, 97, 98%, 99%, or 100% sequence identity to any one of SEQ ID NO: 2. In some embodiments, the nucleic acid molecules encode a polypeptide with an amino acid sequence having at least about 80%, 90%, 95%, 96%, 97, 98%, 99%, or 100% sequence identity to any one of SEQ ID NO: 3. In some embodiments, the nucleic acid molecules encode a polypeptide with an amino acid sequence having at least about 80%, 90%, 95%, 96%, 97, 98%, 99%, or 100% sequence identity to any one of SEQ ID NO: 4. In some embodiments, the nucleic acid molecules encode a polypeptide with an amino acid sequence having at least about 80%, 90%, 95%, 96%, 97, 98%, 99%, or 100% sequence identity to any one of SEQ ID NO: 5. In some embodiments, the nucleic acid molecules encode a polypeptide with an amino acid sequence having at least about 80%, 90%, 95%, 96%, 97, 98%, 99%, or 100% sequence identity to any one of SEQ ID NO: 6.

The nucleic acid sequences encoding the chimeric receptors can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, J. Sambrook & D. W. Russell (2012). Molecular Cloning: A Laboratory Manual (4th ed.). Cold Spring Harbor, N.Y.: Cold Spring Harbor Laboratory and J. Sambrook & D. W. Russell (2001). Molecular Cloning: A Laboratory Manual (3rd ed.). Cold Spring Harbor, N.Y.: Cold Spring Harbor Laboratory (jointly referred to herein as “Sambrook”); F. M. Ausubel (1987). Current Protocols in Molecular Biology. New York, N.Y.: Wiley (including supplements through 2014); D. M. Bollag et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; K. B. Mullis et al., (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B. V., the disclosures of which are incorporated herein by reference.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acids of the present disclosure can be introduced into a host cell, such as a human T lymphocyte, to produce a recombinant cell containing the nucleic acid molecule. Accordingly, some embodiments of the disclosure relate to a methods for making recombinant cells, including: (a) providing a cell capable of protein expression and (b) contacting the provided cell with a recombinant nucleic acid of the disclosure.

Introduction of the nucleic acid molecules of the disclosure into cells can be achieved by viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas9, DNA-guided endonuclease genome editing NgAgo (Natronobacterium gregoryi Argonaute), or TALENs genome editing (transcription activator-like effector nucleases). In some embodiments, the nucleic acid molecule present in the recombinant host cell as a mini-circle expression vector for stable or transient expression.

The nucleic acid molecules can be encapsulated in a viral capsid or a lipid nanoparticle. Alternatively, endonuclease polypeptide(s) can be delivered by viral or non-viral delivery vehicles known in the art, such as electroporation or lipid nanoparticles. For example, introduction of nucleic acids into cells may be achieved using viral transduction methods. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also amenable for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell, a cytotoxic T cell, or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a human subject.

In some embodiments, the recombinant cell further includes a second nucleic acid molecule, wherein the first nucleic acid molecule and the second nucleic acid molecule do not have the same sequence. In some embodiments, the recombinant cell further includes a second chimeric polypeptide, wherein the first chimeric polypeptide and the second chimeric polypeptide do not have the same sequence. In some embodiments, the first chimeric polypeptide modulates the expression and/or activity of the second chimeric polypeptide.

In some embodiments, the recombinant cell further includes an expression cassette encoding a protein of interest operably linked to a promoter, wherein expression of the protein of interest is modulated by the chimeric receptor transcription regulator. In some embodiments, the protein of interest is heterologous to recombinant cell. In principle, there are no particular limitations with regard to suitable proteins whose expression is modulated by the chimeric receptor transcription regulator. Non-limiting examples of proteins suitable for the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporters. In some embodiments, the immune cell receptor is a T-cell receptor (TCR). In some embodiments, the immune cell receptor is a chimeric antigen receptor (CAR). In some embodiments, the expression cassette encoding the protein of interest is incorporated into the same nucleic acid molecule that encodes the Fn Notch receptor of the disclosure. In some embodiments, the expression cassette encoding the protein of interest is incorporated into a second expression vector that is separate from the nucleic acid molecule encoding the Fn Notch receptor of the disclosure.

In another aspect, provided herein are various cell cultures including at least one recombinant cell as disclosed herein, and a culture medium. Generally, the culture medium can be one of many suitable culture media for the cell cultures described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the chimeric receptors of the disclosure can also be administered by transfection or infection using methods known in the art, including but not limited to the methods described in McCaffrey et al., Nature (2002) 418:6893; Xia et al., Nature Biotechnol (2002) 20:1006-10; or Putnam, Am J Health Syst Pharm (1996) 53:151-60 (erratum at Am J Health Syst Pharm (1996) 53:325).

### Methods of the Disclosure

Administration of any one of the therapeutic compositions described herein, e.g., nucleic acids, recombinant cells, and pharmaceutical compositions, can be used to treat individuals in the treatment of relevant health conditions or diseases, such as cancers and chronic infections. In some embodiments, the nucleic acids, recombinant cells, and pharmaceutical compositions described herein can be incorporated into therapeutic agents for use in methods of treating an individual who has, who is suspected of having, or who may be at high risk for developing one or more autoimmune disorders or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Accordingly, in one aspect, some embodiments of the disclosure relate to methods for inhibiting an activity of a target cell in an individual, the methods include administering to the individual a first therapy including one or more of nucleic acids, recombinant cells, and pharmaceutical compositions as disclosed herein, wherein the first therapy inhibits a measurable activity of the target cell. For example, an activity of the target cell may be inhibited if its proliferation is reduced, if its pathologic or pathogenic behavior is reduced, if it is destroyed or killed, and the like. Inhibition includes a reduction of the measured activity of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cell as disclosed herein, wherein the recombinant cell inhibits an activity of the target cell in the individual. Generally, the target cell of the disclosed methods can be any cell and can be, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In another aspect, some embodiments of the disclosure relate to methods for the treatment of a health condition (e.g., disease) in an individual in need thereof, the methods include administering to the individual a first therapy including one or more of chimeric polypeptides, Fn Notch receptors, nucleic acids, recombinant cells, and pharmaceutical compositions as disclosed herein, wherein the first therapy treats the health condition in the individual. In some embodiments, the methods include administering to the individual a first therapy including an effective number of the recombinant cell an effective number of the recombinant cell as disclosed herein, wherein the recombinant cells treat the health condition.

In another aspect, some embodiments of the disclosure relate to methods for the assisting in the treatment of a health condition (e.g., disease) in an individual in need thereof, the methods including administering to the individual a first therapy including one or more of chimeric polypeptides, Fn Notch receptors, nucleic acids, recombinant cells, and pharmaceutical compositions as disclosed herein, and a second therapy, wherein the first and second therapies together treat the health condition in the individual. In some embodiments, the methods include administering to the individual a first therapy including an effective number of the recombinant cells as disclosed herein, wherein the recombinant cell treats the health condition.

Administration of Recombinant Cells into an Individual

In some embodiments, the methods of the disclosure involve administering an effective amount of the recombinants cells of the disclosure into an individual who is in need of such method. This administering step can be accomplished using any method of implantation known in the art. For example, the recombinants cells can be injected directly in the individual's blood or otherwise administered to the individual.

In some embodiments, the methods disclosed herein include administering, which term can be used interchangeably with the terms “introducing” and “transplanting,” recombinant cells into an individual, by a method or route that results in at least partial localization of the introduced cells at a desired site such that a desired effect(s) is produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the life time of the individual, i.e., long-term engraftment.

When provided prophylactically, the recombinant cells described herein can be administered to an individual in advance of any symptom of a disease or condition to be treated. Accordingly, in some embodiments the prophylactic administration of a recombinant stem cell population serves to prevent the occurrence of symptoms of the disease or condition.

When provided therapeutically in some embodiments, recombinant stem cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments described herein, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A cell composition can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation, and the like. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, in other words a population of recombinant cells are administered other than directly into a target site, tissue, or organ, such that it enters, instead, the individual's circulatory system and, thus, is subject to metabolism and other like processes.

The efficacy of a treatment of the disclosure can be determined by the skilled clinician. However, one skilled in the art will appreciate that a treatment is considered effective treatment if any one or all of the signs or symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of disease can be determined by the skilled clinician. However, a treatment is considered effective treatment if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments of the disclosed methods, the individual is a mammal. In some embodiments, the mammal is human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

### Additional Therapies

As discussed supra, the recombinant cells, and pharmaceutical compositions described herein can be administered in combination with one or more additional therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

### Methods for Modulating an Activity of a Cell

In another aspect, provided herein are various methods for modulating an activity of a cell. The methods involve: (a) providing a recombinant cell of the disclosure, and (b) contacting it with a selected ligand, wherein binding of the selected ligand to the extracellular binding domain induces cleavage of a ligand-inducible proteolytic cleavage site and releases the transcription regulator, wherein the released transcription regulator modulates an activity of the recombinant cell. One skilled in the art upon reading the present disclosure will appreciate that the disclosed methods can be carried out in vivo, ex vivo, or in vitro.

Activities of a cell that can be modulated using a method of the present disclosure include, but are not limited to, expression of a selected gene of the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell.

In some embodiments, the released transcription regulator modulates expression of a gene product of the cell. In some embodiments, the released transcription regulator modulates expression of a heterologous gene product in the cell. A heterologous gene product is one that is not normally produced by the cell. For example, the cell can be genetically modified with a nucleic acid including a nucleotide sequence encoding the heterologous gene product.

In some embodiments, the heterologous gene product is a secreted gene product. In some embodiments, the heterologous gene product is a cell surface gene product. In some cases, the heterologous gene product is an intracellular gene product. In some embodiments, the released transcription regulator simultaneously modulates expression of two or more heterologous gene products in the cell.

In some embodiments, the heterologous gene product in the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, an RNA guided nuclease, a site-specific nuclease, a T cell receptor (TCR), a chimeric antigen receptor (CAR), a toxin, a toxin derived protein, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

In some embodiments, the released transcription regulator modulates differentiation of the cell, and wherein the cell is an immune cell, a stem cell, a progenitor cell, or a precursor cell.

The chimeric receptors of the disclosure provide a higher degree of expression than a standard SynNotch receptor, when using identical binding domains and ICDs. Depending on the ligand/binding domain pair and their affinity, the Fn Notch can provide expression enhancement of about 10%, about 15%, about 20%, about 25%, about 30%, about 35%, about 40%, about 45%, or about 50% higher than a corresponding SynNotch receptor.

Additionally, the chimeric receptors of the disclosure can provide transcriptional regulation that responds to the degree of T cell activation, independent of ligand binding. This permits additional flexibility in use, for example in cases where it is desired to enhance or suppress a T cell response when activated despite the absence of the chimeric receptor ligand.

### Systems and Kits

Also provided herein are systems and kits including the chimeric polypeptides, Fn Notch receptors, recombinant nucleic acids, recombinant cells, or pharmaceutical compositions provided and described herein as well as written instructions for making and using the same. For example, provided herein, in some embodiments, are systems and/or kits that include one or more of: a chimeric polypeptide receptor as described herein, a recombinant nucleic acids as described herein, a recombinant cell as described herein, or a pharmaceutical composition as described herein. In some embodiments, the systems and/or kits of the disclosure further include one or more syringes (including pre-filled syringes) and/or catheters (including pre-filled syringes) used to administer one any of the provided recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, killing a target cancer cell, or treating a health condition (e.g., disease) in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g. CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

### Design and Construction of Chimeric Receptor and Response Element Constructs

This Example describes the design and construction of a family of chimeric Fn Notch receptors. Detailed information for various exemplary receptors of the disclosure can be found in Tables 1 and 2 below.

Table 1 provides a brief description for each of the chimeric Notch receptors and the respective components (with components separated by commas). Unless otherwise noted, the entry refers to a protein of human origin. In each construct below, the STS is from Notch 1 (SEQ ID NO:14), and the TF is Gal4, VP64 (SEQ ID NO:15). (m=mouse; h=human)

TABLE 1
Receptor Components

| Construct ID | Receptor | Description | ECD | LP | TMD |
|---|---|---|---|---|---|
| pRay207 | Full Robo | “Full Robo” - antiCD19 | CD8a signal | Robo1 ECD | mNotch1 |
| | | scFv with Robo1 JMD | peptide, myc- | through Fn | |
| | | through the Fn repeats | tag, anti-CD19 | domains | |
| | | | scFv | | |
| pIZ300 | Fn-Robo-m | antiCD19 scFv with | CD8a signal | truncated Robo1 | mNotch1 |
| | | Robo1 ECD through Fn | peptide, myc- | Fn | |
| | | domain, C-terminal portion | tag, anti-CD19 | | |
| | | truncated | scFv | | |
| pIZ311 | Fn Robo- | antiCD19 scFv with | CD8a signal | Robo1 Fn with | mNotch1 |
| | GGS-m | (GGS)₃ linker and Robo1 | peptide, myc- | synthetic | |
| | | Fn domain only | tag, anti-CD19 | (GGS)₃ linker | |
| | | | scFv | | |
| pIZ316 | Fn Notch-m | LaG17 with Robo1 ECD | CD8a signal | truncated Robo1 | mNotch1 |
| | with LaG17 | through Fn domain, C- | peptide, myc- | Fn | |
| | | terminal portion truncated | tag, LaG17 | | |
| | | | nanobody | | |
| pIZ325 | Fn-Robo- | antiCD19 scFv with | CD8a signal | Robo1 Fn with | Notch1 |
| | Notch-h | Robot Fn domain, and C- | peptide, myc- | 9 aa of Notch | |
| | | terminal 9 aa of Notch1 | tag. anti-CD19 | JMD | |
| | | ECD | scFv | | |
| pIZ345 | Fn-Robo-h | antiCD19 scFv with | CD8a signal | truncated Robo1 | Notch1 |
| | | Robo1 ECD through Fn | peptide, myc- | Fn | |
| | | domain, C-terminal portion | tag, anti-CD19 | | |
| | | truncated, with hNotch1 | scFv | | |
| | | TMD | | | |

Table 2 provides a brief description for each of the chimeric Notch receptors, their corresponding components, as well as corresponding sequence identifiers as set forth in the Sequence Listing. ECD: extracellular domain; LP: linking polypeptide; TMD: transmembrane domain; STS: stop-transfer-sequence; TF: transcriptional factor.

TABLE 2
Component Sequences

| Construct | Receptor | | | | | | Full |
|---|---|---|---|---|---|---|---|
| ID | Description | ECD | LP | TMD | STS | TF | sequence |
| pRay207 | antiCD19scFv- | SEQ ID | SEQ ID | SEQ ID | SEQ ID | SEQ ID | SEQ ID |
| | Robo1- | NO: 7 | NO: 9 | NO: 12 | NO: 14 | NO: 15 | NO: 1 |
| | mNotch1TMD- | | | | | | |
| | Gal4VP64 | | | | | | |
| pIZ300 | antiCD19scFv- | SEQ ID | SEQ ID | SEQ ID | SEQ ID | SEQ ID | SEQ ID |
| | CD8Hinge2- | NO: 7 | NO: 19 | NO: 12 | NO: 14 | NO: 15 | NO: 2 |
| | Notch1TMD- | | | | | | |
| | Gal4VP64 | | | | | | |
| pIZ311 | antiCD19scFv- | SEQ ID | SEQ ID | SEQ ID | SEQ ID | SEQ ID | SEQ ID |
| | CD28Hinge- | NO: 7 | NO: 10 | NO: 21 | NO: 14 | NO: 15 | NO: 3 |
| | Notch1TMD- | | | | | | |
| | Gal4VP64 | | | | | | |
| pIZ316 | antiCD19scFv- | SEQ ID | SEQ ID | SEQ ID | SEQ ID | SEQ ID | SEQ ID |
| | IgG4Hinge- | NO: 8 | NO: 19 | NO: 12 | NO: 14 | NO: 15 | NO: 4 |
| | Notch1TMD- | | | | | | |
| | Gal4VP64 | | | | | | |
| pIZ325 | antiCD19scFv- | SEQ ID | SEQ ID | SEQ ID | SEQ ID | SEQ ID | SEQ ID |
| | OX40- | NO: 7 | NO: 11 | NO: 13 | NO: 14 | NO: 15 | NO: 5 |
| | Notch1TMD- | | | | | | |
| | Gal4VP64 | | | | | | |
| pIZ345 | antiCD19scFv- | SEQ ID | SEQ ID | SEQ ID | SEQ ID | SEQ ID | SEQ ID |
| | CD8Hinge2- | NO: 7 | NO: 20 | NO: 13 | NO: 14 | NO: 15 | NO: 6 |
| | Notch1TMD- | | | | | | |
| | Notch2STS- | | | | | | |
| | Gal4VP64 | | | | | | |

The chimeric receptors described in Tables 1-2 above were built by fusing a single-chain antigen-binding fragment CD19 scFv (Porter D L et al., 2011) to the corresponding receptor scaffold and a synthetic transcriptional regulator GAL4-VP64. For the construction of these receptors, DNA fragments coding for the amino acid sequences provided in Table 1 and Sequence Listing were PCR amplified from synthesized gene fragments or plasmids containing DNA sequence for the indicated protein, and assembled using standard cloning techniques (e.g., overhang PCR, fusion PCR, and In-fusion cloning) with flanking translation start and stop sequences, into a BamHI cloning site of the lentiviral expression vector pHR-SIN-pGK

The transcriptional regulator GAL4-VP64 used in these experiments contained a DNA domain from yeast GAL4 transcription factor fused to an activation domain VP64, which consists of a tetrameric repeat of the minimal activation domain (amino acids 437-447) of the herpes simplex protein VP16. All receptors contained an N-terminal CD8a signal peptide (MALPVTALLLPLALLLHAARP) (SEQ ID NO: 17) for membrane targeting and a myc-tag (EQKLISEEDL) (SEQ ID NO: 16) for suitable determination of surface expression with an antibody conjugated to a fluorescent dye (α-myc A647®, Cell Signaling Technology, Cat #2233). The receptors were each cloned into a modified lentiviral pHR′SIN:CSW vector (K. T. Roybal et al., Cell (2016) 167(2):419-32) containing a phosphoglycerate kinase (PGK) promoter for all primary T cell experiments described in Examples 3-4 below.

The pHR′SIN:CSW vector was also modified to produce the response element plasmids. For this purpose, five copies of a target sequence for binding of GAL4 DBD domain (GGAGCACTGTCCTCCGAACG) (SEQ ID NO: 18) were cloned 5′ to a minimal pybTATA promoter. Also included in the response element plasmids is a PGK promoter that constitutively drives expression of a yellow fluorescent reporter protein (mCitrine) to suitably identify successfully transduced T cells.

For the construction of all inducible BFP vectors, the coding sequence for a blue fluorescent reporter protein (BFP) was cloned via a BamHI site in the multiple cloning site located 3′ to the GAL4 response elements. For the construction of all inducible CAR vectors, the CARs were tagged C-terminally with a green fluorescent reporter protein (GFP), and were cloned via a BamHI site in the multiple cloning site located 3′ to the GAL4 response elements. All constructs were cloned via cloning kit (In-Fusion® cloning, Clontech #ST0345) according to the manufacturer's instructions.

### Example 2

pPrimary Human T Cell Isolation and Culture

This Example describes the isolation and culture of primary human T cells that were subsequently used in various cell transduction experiments described in Example 3 below.

In these experiments, primary CD4⁺ and CD8⁺ T cells were isolated from blood after apheresis and enriched by negative selection using human T cell isolation kits (human CD4⁺ or CD8⁺ enrichment cocktail; STEMCELL Technologies Cat #15062 and 15063). Blood was obtained from Blood Centers of the Pacific (San Francisco, Calif.) as approved by the University Institutional Review Board. T cells were cryopreserved in growth medium (RPMI-1640, UCSF cell culture core) with 20% human AB serum (Valley Biomedical Inc., #HP1022) and 10% DMSO. After thawing, T cells were cultured in human T cell medium containing X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### Example 3

Human T cells were stably transduced with lentiviral vectors

The Example describes a general protocol used for lentiviral transduction of human T cells.

Generally, lentiviral vectors pseudo-typed with vesicular stomatitis virus envelope G protein (VSV-G) (pantropic vectors) were produced via transfection of Lenti-X™ 293T cells (Clontech #11131D) with a pHR′ SIN:CSW transgene expression vector and the viral packaging plasmids pCMVdR8.91 and pMD2.G using Mirus TransIT®-Lenti (Mirus, #MIR 6606). Generally, primary T cells were thawed the same day and, after 24 hours in culture, were stimulated with beads having anti-CD3 and anti-CD28 antibodies bound to the surface (Human T-Activator CD3/CD28 Dynabeads®, Life Technologies #11131D) at a 1:3 cell:bead ratio. At 48 hours, viral supernatant was harvested and the primary T cells were exposed to the virus for 24 hours. At Day 5 post T cell stimulation, the beads were removed, and the T cells expanded until Day 14 when they were rested and could be used in assays. T cells were sorted for assays with a Beckton Dickinson (BD Biosciences) FACSAria™ II flow cytometer. AND-gate T cells exhibiting basal CAR expression were gated out during sorting.

### Example 4

### Generation of Sender Cells

This Example describes the generation of myelogenous leukemia cells expressing CD19 at equivalent levels as Daudi tumors.

The cancer cell lines used were K562 myelogenous leukemia cells (ATCC #CCL-243) and Daudi B cell lymphoblasts (ATCC #CCL-213). The K562 cells were lentivirally transduced to stably express human CD19 at levels equivalent to Daudi tumors. CD19 levels were determined by staining the cells with α-CD19 APC (Biolegend® #302212). All cell lines were sorted for expression of the transgenes.

### Example 5

### Generation of Reporter Jurkat T Cells

This Example describes the generation of reporter Jurkat T cells that were subsequently used for the screening of transmembrane domains (TMD) and/or stop-transfer sequences (STS).

In these experiments, E6-1 Jurkat T cells (ATCC #TIB-152) were lentivirally transduced with a reporter plasmid carrying an inducible BFP reporter gene and a constitutive mCitrine reporter gene, as described previously (K. T. Roybal et al., Cell (2016) 164:1-10). Reporter-positive Jurkat cells were sorted for mCitrine expression using a Beckton Dickinson (BD Biosciences) FACSAria™ II flow cytometer and expanded.

Lentiviral particles were produced with the receptor transgene expression vector as described previously (L. Morsut et al., Cell (2016) 164:780-91). Reporter-positive Jurkat cells were transduced with individual receptors and expanded for experimentation in 96 well plates.

### Example 6

### Stimulation of Primary T Cells In Vitro

This Example describes experiments performed to demonstrate the stimulation of primary T cells in vitro by the chimeric Fn Notch polypeptides described herein.

For all in vitro T cell stimulations, 1×10⁵ T cells were co-cultured with K562 sender cells (see Example 4) at a 1:1 ratio in flat bottom 96-well tissue culture plates. The cultures were analyzed at 24 hours for reporter activation with a BD Fortessa™ X-50. All flow cytometry analysis was performed in FlowJo™ software (TreeStar, Inc.).

As shown in Table 3, each of the Fn Notch constructs pRay207 (Full Robo), pIZ300 (Fn-Robo-m), pIZ311 (Fn Robo-GGS-m), pIZ316 (Fn Notch-m with LaG17), pIZ325 (Fn-Robo-Notch-h), and pIZ345 (Fn-Robo-h) were able to stimulate primary T cells as determined by expression of BFP reporter gene.

The results of this experiment are summarized in Table 3 below.

TABLE 3
Receptor Characteristics

| | | | Sensitive to T cell |
|---|---|---|---|
| Receptor | Description | “Switch-like” | activation |
| Full Robo | Anti-CD19 scFv with | No - Always ON | No |
| | Robo JMD, mNotch1 | | |
| | TMD | | |
| Fn-Robo-m | Anti-CD19 scFv with | Yes | Slightly |
| | truncated Robo JMD, | | |
| | mNotch1 TMD | | |
| Fn Robo-GGS-m | Anti-CD19 scFv with | Yes | Slightly |
| | Robo Fn domains, | | |
| | (GGS)₃ replacing Robo | | |
| | sequence between TMD | | |
| | and Fn repeats, mNotch1 | | |
| | TMD | | |
| Fn Notch-m with | Anti-LaG17 nanobodv | Yes | Slightly |
| LaG17 | with truncated Robo | | |
| | JMD, mNotch1 TMD | | |
| Fn-Robo-Notch-h | Anti-CD19 scFv with | Yes | Better signal to |
| | Robo Fn domains, Notch | | noise ratio than |
| | (without NNR) replacing | | Fn Notch-Robo |
| | Robo sequence between | | |
| | TMD and Fn repeats, | | |
| | mNotch1 TMD | | |
| Fn-Robo-h | Anti-CD19 scFv with | Yes | Slightly |
| | truncated Robo JMD, | | |
| | hNotch1 TMD | | |

The results demonstrated that receptors with the full Robo JMD were ineffective as receptors, as they were constitutively on. The remaining receptors were constructed with a reduced sequence length between the TMD and the Fn repeats. The receptors Fn-Robo-m, Fn Notch-m with LaG17 (LaG17, anti-GFP is a readily expressible recombinant nanobody which has a relatively low affinity and high specificity against GFP), and Fn-Robo-h each have truncated Robo JMD domains, with seven amino acids between the TMD and Fn repeats. In Fn-Robo-Notch-h, the Robo sequence was replaced with the corresponding Notch sequence (after deletion of the negative regulator region), having nine amino acids between the TMD and Fn repeats. In Fn-Robo-GGS-m, this region was replaced with a fully synthetic sequence, (GGS)₃, having nine amino acids between the TMD and Fn repeats. This also demonstrates that receptors having 7-9 amino acids between the TMD and Fn repeats are active, while receptors having 27 amino acids between the TMD and Fn repeats (Full Robo) are less satisfactory.

### Example 7

### Comparison of Fn Notch and SynNotch

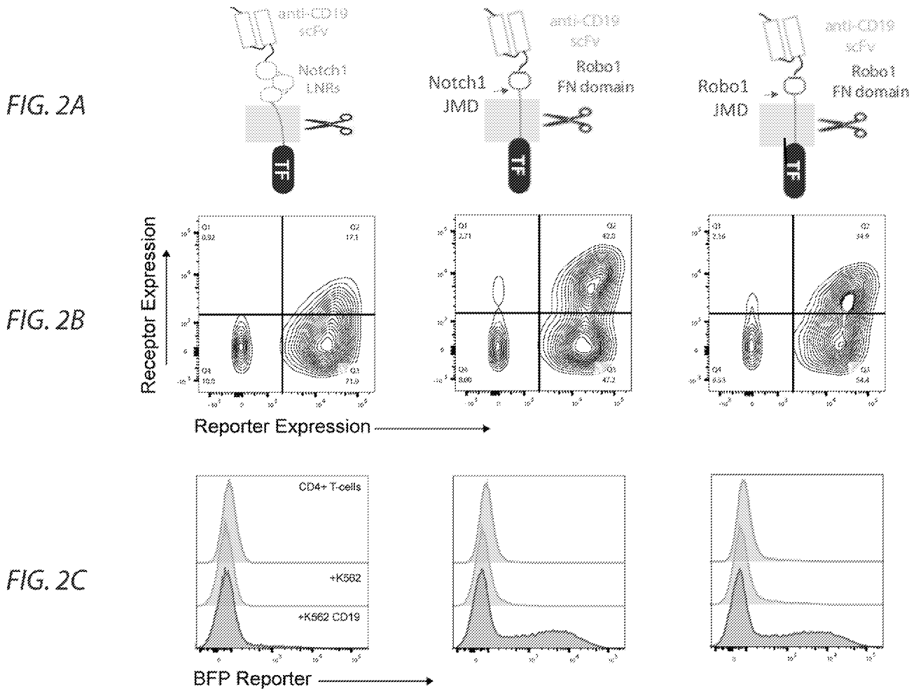

A first generation SynNotch receptor was compared with an Fn Notch receptor having a Robo1 Fn domain instead of the Notch NRR, with the Fn domain linked to the TMD with a polypeptide from Notch1 (lacking the NRR), and an Fn Notch receptor having a Robo1 Fn domain instead of the Notch sequence, including Robo1 sequence between the Fn domain and the TMD (see FIG. 2A).

Primary human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive mCitrine gene located on the reporter plasmid. Double positive cells were sorted for on Day 5 post initial T cell stimulation and expanded further for activation testing. The results are shown in FIG. 2B.

Receptor activation testing without TCR activation was then tested. 1×10⁵ double positive T cells expressing anti-CD19 receptors were co-cultured with: nothing, 1×10⁵ K562 cells or 1×10⁵ CD19+K562 cells for 24 hours with each corresponding receptor. Transcriptional activation of an inducible BFP reporter gene was measured using a Fortessa X-50 (BD Biosciences). The results are shown in FIG. 2C.

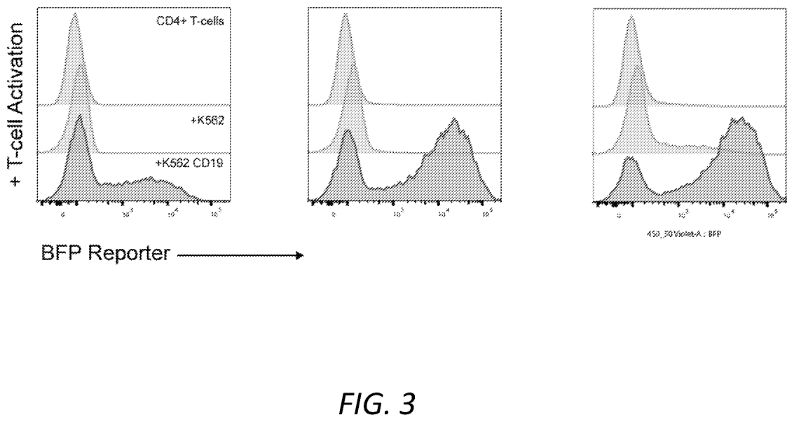

To trigger T cell activation, anti-MCAM, anti-CD3 Bi-specific T cell Engagers (MCAM BiTE®s) were used, which activate the T cell receptor in the presence of (MCAM+) K562 cells. 1×10⁵ double positive T cells expressing anti-CD19 receptors were co-cultured with: MCAM BiTE®s, 1 1×10⁵ E5 K562 cells+MCAM BiTE®s, or 1×10⁵ CD19+K562 cells+MCAM BiTE®s for 24 hours. Transcriptional activation of an inducible BFP reporter gene was measured using a Fortessa X-50 (BD Biosciences). The results are shown in FIG. 3.

### Example 8

### Linking Polypeptide Substitutions

This experiment was performed to determine the effect in Fn Notch receptors of varying the linking polypeptide. Exemplified here as linking polypeptides are: Robo1 sequence, (GGS)₃, (GSS)₂, (GSS)₁, and none (a direct bond between the Robo Fn repeats and TMD).

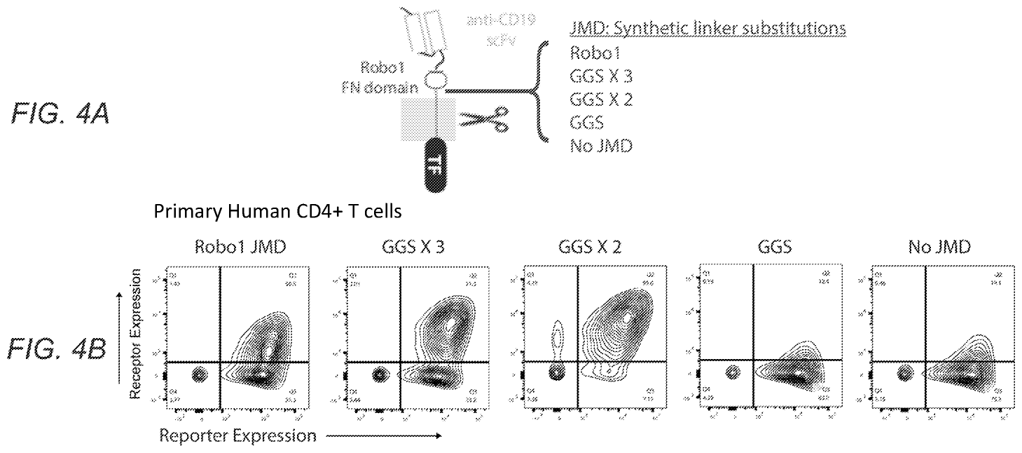

Primary human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive fluorescent protein located on the reporter plasmid. Double positive cells were sorted for on Day 5 post initial T cell stimulation and expanded further for activation testing. FIG. 4B shows the flow cytometry data of receptor expression for each variation.

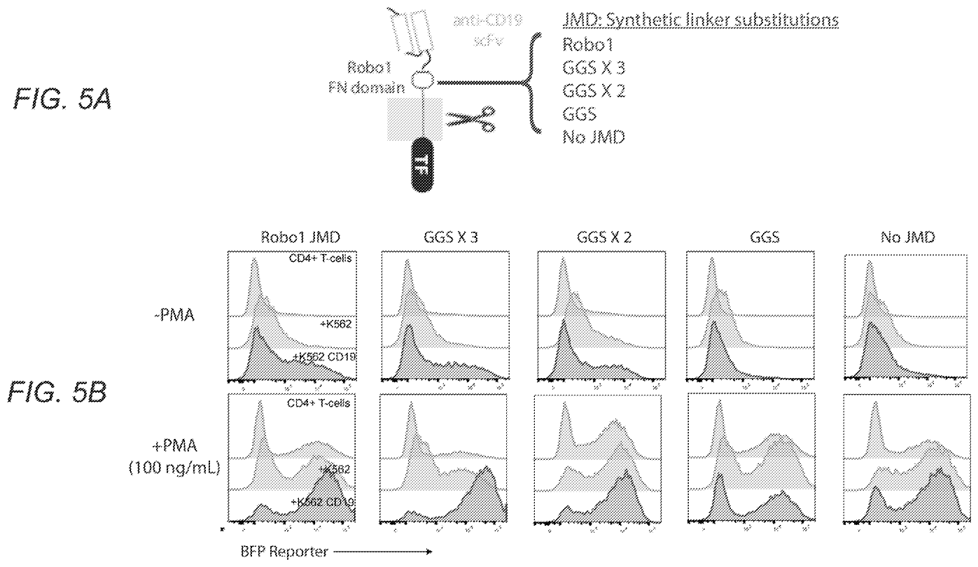

1×10⁵ double positive T cells expressing anti-CD19 receptors were co-cultured with: nothing, 1×10⁵ K562 cells, or 1×10⁵ CD19+K562 cells for 24 hours. Transcriptional activation of an inducible BFP reporter gene was measured using a Fortessa X-50 (BD Biosciences). FIG. 5A shows receptor activation testing without TCR activation.

Phorbol 12-myristate 13-acetate (PMA), a diacyl glycerol analog, was added to all cultures and co-cultures to trigger PKC signaling. FIG. 5B shows receptor activation with TCR activation.

### Example 9

### Ligand Binding Domains

This experiment was performed to illustrate Fn Notch receptors having different ligand binding domains. Fn Notch receptors were constructed as described above, substituting an anti-GFP LagG17 nanobody or an anti-ALPPL2 scFv instead of the anti-CD19 scFv ligand binding domains.

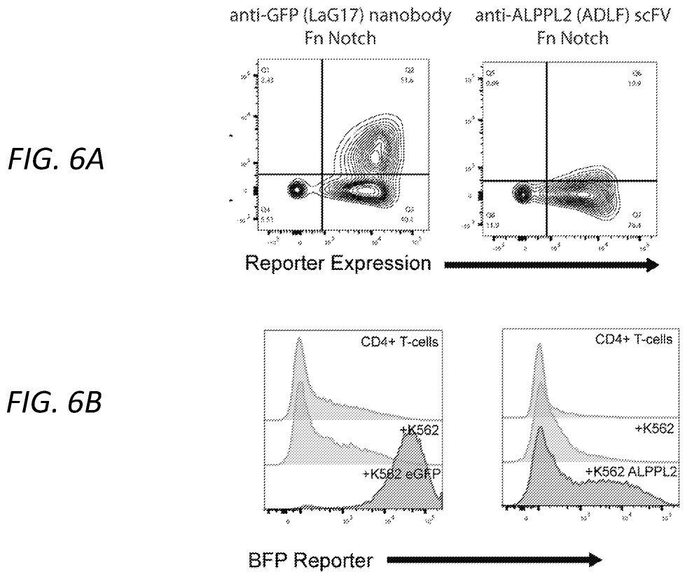

Primary CD4 human T cells were activated with anti-CD3/anti-CD28 Dynabeads (Gibco) and transduced with two lentiviral constructs expressing either a receptor or a transcriptional reporter construct. Receptor expression was measured using an AlexaFluor647-tagged anti-myc antibody (Cell Signaling). Reporter expression was measured through a constitutive fluorescent protein found on the reporter plasmid. Double positive cells were sorted for on Day 5 post initial T cell stimulation and expanded further for activation testing. The results are shown in FIG. 6A.

1×10⁵ double positive CD8+ T cells expressing anti-GFP or anti-ALPPL2 Fn Notch were co-cultured with: nothing, 1×10⁵ K562 cells, or 1×10⁵ surface GFP K562 cells/ALPPL2+ K562 cells for 24 hours. Transcriptional activation of an inducible BFP reporter gene was measured using a Fortessa X-50 (BD Biosciences). FIG. 6B shows flow cytometry data for receptor activation.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

David L. Porter, M. D., Bruce L. Levine, Ph.D., Michael Kalos, Ph.D., Adam Bagg, M. D., and Carl H. June, M. D. Chimeric Antigen Receptor—Modified T Cells in Chronic Lymphoid Leukemia. N. Engl J Med. 2011 Aug. 25; 365(8): 725-733.
Morsut L, Roybal K T, Xiong X, Gordley R M, Coyle S M, Thomson M, and LimWA. Engineering Customized Cell Sensing and Response Behaviors Using Synthetic Notch Receptors. Cell. 2016 Feb. 11; 164(4): 780-791.
Naso M F, Tomkowicz B, Perry W L 3rd, Strohl W R. Adeno-Associated Virus (AAV) as a Vector for Gene Therapy. BioDrugs. 2017; 31(4):317-334.
Nasri M, Karimi A, Allahbakhshian Farsani M. Production, purification and titration of a lentivirus-based vector for gene delivery purposes. Cytotechnology. 2014; 66(6):1031-1038.
Roybal K T, Jasper Z. Williams, Leonardo Morsut, Levi J. Rupp, Isabel Kolinko, Joseph H. Choe, Whitney J. Walker, Krista A. McNally, and Wendell A. Lim. Engineering T cells with Customized Therapeutic Response Programs Using Synthetic Notch Receptors. Cell. 2016 Oct. 6; 167(2): 419-432.
Samulski and Muzyczka (2014). AAV-Mediated Gene Therapy for Research and Therapeutic Purposes. Annu. Rev. Virol. 1,427.
Sakuma, et al. (2012). Lentiviral vectors: basic to translational. Biochem. J. 443,603.
Watson D. J., Wolfe J. H. Viral vectors for gene therapy: methods and protocols. Totowa, N.J., USA: Humana Press; 2003. pp. 383-404.

What is claimed is:

1. A chimeric polypeptide comprising, from N-terminus to C-terminus:
a) an extracellular ligand binding domain (ECD) having a binding affinity for a selected ligand;
b) a linking polypeptide comprising one, two, or three Robo1 fibronectin (Fn) repeats and a short sequence of from about two to about 20 amino acids;
c) a transmembrane domain (TMD) comprising one or more ligand-inducible proteolytic cleavage sites; and
d) an intracellular domain (ICD) comprising a transcription regulator, wherein binding of the selected ligand to the extracellular binding domain induces cleavage at the ligand-inducible proteolytic cleavage site between the transcription regulator and the linking polypeptide.

2. The chimeric polypeptide of claim 1, wherein the chimeric polypeptide does not comprise a LIN-12-Notch repeat (LNR) and/or a heterodimerization domain (HD) of a Notch receptor.

3. The chimeric polypeptide of claim 1 or 2, further comprising a stop-transfer sequence (STS) between the TMD and the ICD.

4. The chimeric polypeptide of any one of claims 1 to 3, wherein the linking polypeptide comprises two Fn repeats.

5. The chimeric polypeptide of any one of claims 1 to 4, wherein the linking polypeptide comprises one Fn repeat.

6. The chimeric polypeptide of any one of claims 1 to 5, wherein the short sequence has at least about 80% sequence identity to a Robo1 juxtamembrane domain (JMD).

7. The chimeric polypeptide of claim 6, wherein the short sequence has at least about 90% sequence identity to a Robo1 JMD.

8. The chimeric polypeptide of claim 6, wherein the short sequence has at least about 95% sequence identity to a Robo1 JMD.

9. The chimeric polypeptide of claim 6, wherein the short sequence has at least about 98% sequence identity to a Robo1 JMD.

10. The chimeric polypeptide of any one of claims 1 to 5, wherein the short sequence has at least about 80% sequence identity to a Notch1, Notch2, Notch3, or Notch4 juxtamembrane domain (JMD).

11. The chimeric polypeptide of claim 10, wherein the short sequence has at least about 90% sequence identity to a Notch1, Notch2, Notch3, or Notch4 JMD.

12. The chimeric polypeptide of claim 10, wherein the short sequence has at least about 95% sequence identity to a Notch1, Notch2, Notch3, or Notch4 JMD.

13. The chimeric polypeptide of claim 10, wherein the short sequence has at least about 98% sequence identity to a Notch1, Notch2, Notch3, or Notch4 JMD.

14. The chimeric polypeptide of any one of claims 1 to 5, wherein the short sequence has less than about 80% sequence identity to a Robo1, Notch1, Notch2, Notch3, or Notch4 JMD.

15. The chimeric polypeptide of claim 14, wherein the short sequence comprises a Gly-Ser polymer.

16. The chimeric polypeptide of claim 14, wherein the short sequence comprises a (GGS)ₙ polymer, where n is an integer from 1 to 50.

17. The chimeric polypeptide of claim 14, wherein the short sequence comprises a GGS, (GGS)₂, (GGS)₃, (GGS)₃, (GGS)₉, (GGS)₁₂, (GGS)₁₅, or (GGS)₁₈ polymer.

18. The chimeric polypeptide of any one of claims 1 to 17, wherein the ECD comprises an antigen-binding moiety capable of binding to a ligand on the surface of a cell.

19. The chimeric polypeptide of claim 18, wherein the cell is a pathogen.

20. The chimeric polypeptide of any one of claims 1 to 19, wherein the ligand comprises a protein or a carbohydrate.

21. The chimeric polypeptide of any one of claims 1 to 20, wherein the ligand is selected from the group consisting of ALPPL2, BCMA, GFP, eGFP, SIRPα, CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE.

22. The chimeric polypeptide of any one of claims 1 to 21, wherein the ligand is selected from cell surface receptors, adhesion proteins, integrins, mucins, lectins, tumor associated antigens, and tumor-specific antigens.

23. The chimeric polypeptide of any one of claims 1 to 22 wherein the ligand is a tumor-associated antigen or a tumor-specific associated antigen.

24. The chimeric polypeptide of any one of claims 1 to 23, wherein the ECD comprises the ligand-binding portion of a receptor.

25. The chimeric polypeptide of claim 18, wherein the antigen-binding moiety is selected from the group consisting of an antibody, a nanobody, a diabody, a triabody, or a minibody, a F(ab′)2 fragment, a Fab fragment, a single chain variable fragment (scFv) or a single domain antibody (sdAb), or a functional fragment thereof.

26. The chimeric polypeptide of claim 25, wherein the antigen-binding moiety comprises an scFv.

27. The chimeric polypeptide of any one of claims 25 to 26, wherein the antigen-binding moiety is a tumor-associated antigen selected from the group consisting of ALPPL2, CD19, B7H3 (CD276), BCMA, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-beta, PRSS21, PSCA, PSMA, ROR1, SSEA-4, TAG72, TEM1/CD248, TEM7R, TSHR, VEGFR2, BCMA (CD269), ALPI, citrullinated vimentin, cMet, and Axl.

28. The chimeric polypeptide of claim 27, wherein the tumor-associated antigen is ALPPL2, CD19, CEA, HER2, MUC1, CD20, or EGFR.

29. The chimeric polypeptide of claim 28, wherein the tumor-associated antigen is CD19.

30. The chimeric polypeptide of any one of claims 1 to 29, wherein the ligand-inducible proteolytic cleavage site is a γ secretase cleavage site.

31. The chimeric polypeptide of any one of claims 1 to 30, wherein the transcription regulator comprises a transcriptional activator, or a transcriptional repressor.

32. The chimeric polypeptide of any one of claims 1 to 31, wherein the ICD comprises a nuclear localization sequence and a transcription regulator sequence selected from Gal4-VP16, Gal4-VP64, tetR-VP64, ZFHD1-VP64, Gal4-KRAB, and HAP1-VP16.

33. The chimeric polypeptide of any one of claims 1 to 32, further comprising a signal sequence, a detectable label, a tumor-specific cleavage site, a disease-specific cleavage site, or a combination thereof.

34. The chimeric polypeptide of any one of claims 3 to 19, wherein the STS comprises an amino acid sequence having at least 80% sequence identity to SEQ ID NO: 14.

35. The chimeric polypeptide of any one of claims 1 to 20, wherein the linking polypeptide comprises an amino acid sequence having at least 80% sequence identity to any one of SEQ ID NOS: 9-11 and 19-20.

36. The chimeric polypeptide of claim 35, wherein the linking polypeptide comprises an amino acid sequence having at least 90% sequence identity to any one of SEQ ID NOS: 9-11 and 19-20.

37. The chimeric polypeptide of claim 36, wherein the linking polypeptide comprises an amino acid sequence having at least 95% sequence identity to any one of SEQ ID NOS: 9-11 and 19-20.

38. The chimeric polypeptide of claim 37, wherein the linking polypeptide comprises an amino acid sequence substantially identical to any one of SEQ ID NOS: 9-11 and 19-20.

39. The chimeric polypeptide of any one of claims 1 to 38, wherein the TMD comprises an amino acid sequence having at least 80% sequence identity to either of SEQ ID NOS: 12-13 and 21.

40. The chimeric polypeptide of claim 39, wherein the TMD comprises an amino acid sequence having at least 90% sequence identity to either of SEQ ID NOS: 12-13 and 21.

41. The chimeric polypeptide of any one of claims 1 to 40, wherein the TMD comprises an amino acid sequence having at least 95% sequence identity to either of SEQ ID NOS: 12-13 and 21.

42. The chimeric polypeptide of claim 41, wherein the TMD comprises an amino acid sequence substantially identical to either of SEQ ID NOS: 12-13 and 21.

43. The chimeric polypeptide of any one of claims 1 to 42, wherein:
a) the linking polypeptide comprises an amino acid sequence having at least 80% sequence identity to any one of SEQ ID NOS: 9-11 and 19-20;
b) the TMD comprises an amino acid sequence having at least 80% sequence identity to any one of SEQ ID NO: 12-13 and 21; and
c) the STS comprises an amino acid sequence having at least 80% sequence identity to SEQ ID NO: 14.

44. The chimeric polypeptide of any one of claims 1 to 43, wherein the chimeric polypeptide comprises an amino acid sequence having at least 80% sequence identity to any one of SEQ ID NOS: 1-6.

45. A recombinant nucleic acid comprising a nucleotide sequence encoding a chimeric polypeptide according to any one of claims 1 to 44.

46. The recombinant nucleic acid of claim 45, wherein the nucleotide sequence is incorporated into an expression cassette or an expression vector.

47. The recombinant nucleic acid of claim 46, wherein the expression vector is a viral vector.

48. The recombinant nucleic acid of claim 47, wherein the viral vector is a lentiviral vector, an adenovirus vector, an adeno-associated virus vector, or a retroviral vector.

49. A recombinant cell comprising:
a) a chimeric polypeptide according to any one of claims 1 to 44; and/or
b) a recombinant nucleic acid according to any one of claims 45 to 48.

50. The recombinant cell of claim 49, wherein the cell is a mammalian cell.

51. The recombinant cell of claim 50, wherein the mammalian cell is an immune cell, a neuron, an epithelial cell, and endothelial cell, or a stem cell.

52. The recombinant cell of claim 51, wherein the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

53. The recombinant cell of any one of claims 49 to 52, further comprising:
a) a second chimeric polypeptide according to any one of claims 1 to 44; and/or
b) a second nucleic acid according to any one of claims 45 to 48;
wherein the chimeric polypeptide and the second chimeric polypeptide do not have the same sequence, and/or the nucleic acid or the second nucleic acid do not have the same sequence.

54. The recombinant cell of claim 53, wherein the chimeric polypeptide modulates the expression and/or activity of the second chimeric polypeptide.

55. The recombinant cell of any one of claims 49 to 54, further comprising:
an expression cassette encoding a protein of interest operably linked to a promoter, wherein expression of the protein of interest is modulated by the chimeric receptor transcriptional regulator.

56. The recombinant cell of claim 55, wherein the protein of interest is heterologous to the cell.

57. The recombinant cell of claim 55 or 56, wherein the promoter is GAL4.

58. The recombinant cell of claim 55 or 56, wherein the protein of interest is a cytokine, a cytotoxin, a chemokine, an immunomodulator, a pro-apoptotic factor, an anti-apoptotic factor, a hormone, a differentiation factor, a dedifferentiation factor, an immune cell receptor, or a reporter.

59. A cell culture comprising a recombinant cell according to any one of claims 49 to 58, and a culture medium.

60. A pharmaceutical composition comprising a pharmaceutically acceptable carrier, and one or more of the following:
a) a recombinant nucleic acid according to any one of claims 45 to 48; or
b) a recombinant cell according to any one of claims 49 to 58.

61. The pharmaceutical composition of claim 60, wherein the composition comprises a recombinant nucleic acid according to any one of claims 45 to 48, and a pharmaceutically acceptable carrier.

62. The pharmaceutical composition of claim 61, wherein the recombinant nucleic acid is encapsulated in a viral capsid or a lipid nanoparticle.

63. A method for modulating an activity of a cell, the method comprising:
a) providing a recombinant cell according to any one of claims 49 to 58; and
b) contacting the recombinant cell with the selected ligand, wherein binding of the selected ligand to the ECD induces cleavage of a ligand-inducible proteolytic cleavage site and releases the transcription regulator, wherein the released transcription regulator modulates an activity of the recombinant cell.

64. The method of claim 63, the contacting is carried out in vivo, ex vivo, or in vitro.

65. The method of any one of claims 63 to 64, wherein the activity of the cell is selected from the group consisting of: expression of a selected gene in the cell, proliferation of the cell, apoptosis of the cell, non-apoptotic death of the cell, differentiation of the cell, dedifferentiation of the cell, migration of the cell, secretion of a molecule from the cell, cellular adhesion of the cell, and cytolytic activity of the cell.

66. The method of any one of claims 63 to 65, wherein the released transcription regulator modulates expression of a gene product of the cell.

67. The method of any one of claims 63 to 66, wherein the released transcription regulator modulates expression of a heterologous gene product.

68. The method of any one of claims 63 to 67, wherein the gene product of the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T cell receptor, a toxin, a toxin derived protein, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

69. The method of any one of claims 63 to 68, wherein the released transcription regulator modulates differentiation of the cell, and wherein the cell is an immune cell, a stem cell, a progenitor cell, or a precursor cell.

70. A method for inhibiting a target cell in an individual, the method comprising administering to the individual an effective number of the recombinant cell according to any one of claims 49 to 58, wherein the recombinant cell inhibits the target cell in the individual.

71. The method of claim 70, wherein the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T cell lymphoma cell, a renal cancer cell, a sarcoma cell, a stomach cancer cell, a carcinoma cell, a mesothelioma cell, or a sarcoma cell.

72. The method of claim 70, wherein the target cell is a pathogenic cell.

73. A method for the treatment of a health condition in an individual in need thereof, the method comprising: administering to the individual a first therapy comprising an effective number of the recombinant cell according to any one of claims 49 to 58, wherein the recombinant cell treats the health condition in the individual.

74. The method of claim 73, further comprising administering to the individual a second therapy.

75. The method of claim 74, wherein the second therapy is selected from the group consisting of chemotherapy, radiotherapy, immunotherapy, hormonal therapy, or toxin therapy.

76. The method of any one of claims 73 to 75, wherein the first therapy and the second therapy are administered together, in the same composition or in separate compositions.

77. The method claim 76, wherein the first therapy and the second therapy are administered at the same time.

78. The method of any one of claims 74 to 75, wherein the first therapy and the second therapy are administered sequentially.

79. The method of claim 78, wherein the first therapy is administered before the second therapy.

80. The method of claim 78, wherein the first therapy is administered after the second therapy.

81. The method of claim 78, wherein the first therapy and the second therapy are administered in rotation.

82. A system for modulating an activity of a cell, inhibiting a target cancer cell, or treating a health condition in an individual in need thereof, wherein the system comprises one or more of the following:
a) a chimeric polypeptide according to any one of claims 1 to 44;
b) a recombinant nucleic acid according to any one of claims 45 to 48;
c) a recombinant cell according to any one of claims 49 to 58; and
d) a pharmaceutical composition according to any one of claims 60 to 62.

83. A method for making the recombinant cell according to any one of claims 49 to 58, comprising:
a) providing a cell capable of protein expression; and
b) contacting the provided cell with a recombinant nucleic acid according to any one of claims 45 to 48.

84. The method of claim 83, wherein the cell is obtained by leukapheresis of a sample obtained from a human subject, and the cell is contacted ex vivo.

85. The method of claim 83, wherein the recombinant nucleic acid is encapsulated in a viral capsid or a lipid nanoparticle.

86. The use of one or more of the following for the treatment of a health condition:
a) a chimeric polypeptide according to any one of claims 1 to 44;
b) a recombinant nucleic acid according to any one of claims 45 to 48;
c) a recombinant cell according to any one of claims 49 to 58; and
d) a composition according to any one of claims 60 to 62.

87. The use of claim 86, wherein the health condition is cancer.

88. The use of the invention of any one of claims 1 to 87, for the manufacture of a medicament for the treatment of a health condition.
